SEQUENCE LISTING (24 sequences)

SEQ ID NO: 1 - DNA, 2520 bp, S. enterica subsp. IIIb
cggatgagcggcattttccgcgatgtttccctgctgcataagccgacgacgcagattacggatttccacctccatactcatctgaatgacgactttacccaggcggtgcttgaggcggaagttcgactggctggcggcgtgaacgacgatttgcagctcgtgcttcatctctggcagggagaaacgctggcgggagaggcccacgccacgccgggaagtgaaattattgacgagcgcggcggctacgatgaccgcgccacgctacgcctcaatatcaatcgtccggcgctgtggagcgcggaaacgcctaatctgtatcgggcggtgatccaattgcgtaccgccgatggcgagctgattgaagcggaagcgtgtgacgttggcttccgccaggtccgcatcgataaggggctgttactgctaaatggtaaaccgctactgatccgtgggaccaatcggcatgagcatcaccctgagcgcggccaggtgatggattacgatactatggtgcaggatattttgctgatgaagcagaataactttaatgccgttcgctgctcgcattaccccaatcatccgcagtggtacgccctatgtgaccgctatggtctgtacgtggtggatgaagccaacattgaaacccacggtatgacgccgatgaatcgcctgagcgatgacccggactggctacccgccatgagtcagcgtgtgacgcgcatggtgcagcgcgatcgcaaccatccgagcattatcatctggtcgttgggcaatgaatccgggtatggggctaaccatgacgcgctttaccgctggctgaaagcggaagatccttctcgcccggtgcagtatgagggcggcggcgcggacaccgccgcgacggatattatctgcccgatgtatgcgcgcgttgatcaggatcagcccttcccggctgtgccaaaatggtcaatcaagaagtggctctcgctgcccggcgagcagcgtccgctcattctctgcgaatatgcgcatgcgatgggcaacagctttggcggctttgccaaatactggcaggcgtttcgtcagtatccgcgcctgcaggggggctttgtctgggactgggtagatcagtcgttgattaaatatgacgcggacggcaaaccgtggtctgcctacggcggggattttggcgatacgcccaacgatcgtcagttctgcatgaacgggctggtctttgccgatcgcacgccgcatccggcgctgtatgaagcgaaacacgtgcagcaatttttccagtttcgtttgctgccgggcgaagaacgccgcattgaagtacagagcgaatatctgttccgccacagtgataacgagatcctgcgctggatgctggcgcaggagggcaatcagttggccagtggtgaagttgtgctggatatcgccccgcaggggcgacaaattatcctgctacctgcattcccgcagccggaaaccgcagggcagctttggctgacggtacgcgttgagcaaccgctggcgacaagctggtcggaggccgggcatatcagcgcctggcaacagtggccgttggaagaaaagctctgcgtatcgaaacccactcgcgccagcgttgcgcccgccctgacggtgcgtgacggcgaattctgcgtcacgcaaggtaatttgcgttggcagttctgccgtcagcagggctggcttacacagttctggcgggatgatgaagcgcaattgctgaccccgcttatcgatcaattcacccgtgcgccgcttgataacgatattggcgtcagtgaagcgacacgcattgaccccaatgcctgggtggagcgttggaaggcggcaggacattactgcgcggagccagcgctgttgctgtgtgatgcagacgagctggcggatgcggtgctgataaccaccgcgcatgcctggcaatatcagggggcaacgctgtttatcagccgcaaaacctaccggattgatgatcatggcgaaatgcagatcgacatcggggtggaggttgccagtggaatgccgtacccggcgcggattggcttgagctgtcaacttgcgcaggtgaatgaaagggtggagtggctggggctggggccgcatgagaattatccggatcgcctgagcagcgcttgttttgatcgctggaacctcccgctggacgcgatgtatacgccgtatgtctttccgactgaaaatggcctgcgttgtggtactcgccagttgcgttatggcgcgcaccagtggagcggcgattttcagttcaatattagccgttacagccagcggcaactgatggaaaccagccaccgccatttattgcaggcagaatcaggcgtctggctcaatatcgatggttatcatatgggagtgggcggggatgattcatggagcccgtcggtatcgccggaatttcaactgagcgcccggcattaccactaccagatcgcatggaaataaaatacctcattgggcagattgatcctgcccatttttaatgcaaggaaatccata

SEQ ID NO: 2 - DNA, 20 bp, Artificial sequence
  lacZ target nucleotide sequence for forward primer
cgttttggatggcctaacta

SEQ ID NO: 3 - DNA, 20 bp, Artificial sequence
  lacZ target nucleotide sequence for reverse primer
aggtgaatgaaagggtggag

SEQ ID NO: 4 - DNA, 20 bp, Artificial sequence
  lacZ Forward Primer
gcaaaacctaccggattgat

SEQ ID NO: 5 - DNA, 20 bp, Artificial sequence
  lacZ Reverse Primer
tccacttactttcccacctc

SEQ ID NO: 6 - DNA, 24 bp, Artificial sequence
  Target nucleotide sequence for lacZ probe
gtaccgctttacgtctagctgtag

SEQ ID NO: 7 - DNA, 24 bp, Artificial sequence
  lacZ Probe
catggcgaaatgcagatcgacatc

SEQ ID NO: 8 - DNA, 23 bp, Artificial sequence
  Control forward primer
ctcaccaggagattacaacatgg

SEQ ID NO: 9 - DNA, 23 bp, Artificial sequence
  Control reverse primer
agctcagaccaaaagtgaccatc

SEQ ID NO: 10 - DNA, 22 bp, Artificial sequence
  Control probe
caccgacggcgagaccgacttt

SEQ ID NO: 11 - DNA, 2520 bp, S. enterica subsp. IIIb
cggatgagcggcattttccgcgatgtttccctgctgcataagccgacgacgcagattacggatttccacctccatactcatctgaatgacgactttacccaagcggtgcttgaggcggaagttcgactggctggcggcgtgaacgacgatttgcagctcgtgcttcatctctggcagggagaaacgctggcgggagaggcccacgccacgccgggaagtgaaattattgacgagcgcggcggctacgatgaccgcgccacgctacgcctcaatatcaatcgtccggcgctgtggagcgcggaaacgcctaatctgtatcgggcggtgatccaattgcgtaccgccgatggcgagctgattgaagcggaagcgtgtgacgttggcttccgccaggtccgcatcgataaggggctgttactgctaaatggtaaaccgctactgatccgtgggaccaatcggcatgagcatcaccctgagcgcggccaggtgatggattacgatactatggtgcaggatattttgctgatgaagcagaataactttaatgccgttcgctgctcgcattaccccaatcatccgcagtggtacgccctatgtgaccgctatggtctgtacgtggtggatgaagccaacattgaaacccacggtatgacgccgatgaatcgcctgagcgatgacccggactggctacccgccatgagtcagcgtgtgacgcgcatggtgcagcgcgatcgcaaccatccgagcattatcatctggtcgttgggcaatgaatccgggtatggggctaaccatgacgcgctttatcgctggctgaaagcggaagatccttctcgcccggtgcagtatgagggcggcggcgcggacaccgccgcgacggatattatctgcccgatgtatgcgcgcgttgatcaggatcagcccttcccggctgtgccaaaatggtcaatcaagaagtggctctcgctgcccggcgagcagcgtccgctcattctctgcgaatatgcgcatgcgatgggcaacagctttggcggctttgccaaatactggcaggcgtttcgtcagtatccgcgcctgcaggggggctttgtctgggactgggtagatcagtcgttgattaaatatgacgcggacggcaaaccgtggtctgcctacggcggggattttggcgatacgcccaacgatcgtcagttctgcatgaacgggctggtctttgccgatcgcacgccgcatccggcgctgtatgaagcgaaacacgtgcagcaatttttccagtttcgtttgctgccgggcgaagaacgccgcattgaagtacagagcgaatatctgttccgccacagtgataacgagatcctgcgctggatgctggcgcaggagggcaatcagttggccagtggtgaagttgtgctggatatcgccccgcaggggcgacaaattatcctgctacctgcattcccgcagccggaaaccgcagggcagctttggctgacggtacgcgttgagcaaccgctggcgacaagctggtcggaggccgggcatatcagcgcctggcaacagtggccgttggaagaaaagctctgcgtatcgaaacccactcgcgccagcgttgcgcccgccctgacggtgcgtgacggcgaattctgcgtcacgcaaggtaatttgcgttggcagttctgccgtcagcagggctggcttacacagttctggcgggatgatgaagcgcaattgctgaccccgcttatcgatcaattcacccgtgcgccgcttgataacgatattggcgtcagtgaagcgacacgcattgaccccaatgcctgggtggagcgttggaaggcggcaggacattactgcgcggagccagcgctgttgctgtgtgatgcagacgagctggcggatgcggtgctgataaccaccgcgcatgcctggcaatatcagggggcaacgctgtttatcagccgcaaaacctaccggattgatgatcatggcgaaatgcagatcgacatcggggtggaggttgccagtggaatgccgtacccggcgcggattggcttgagctgtcaacttgcgcaggtgaatgaaagggtggagtggctggggctggggccgcatgagaattatccggatcgcctgagcagcgcttgttttgatcgctggaacctcccgctggacgcgatgtatacgccgtatgtctttccgactgaaaatggcctgcgttgcggtactcgccagttgcgttatggcgcgcaccagtggagcggcgattttcagttcaatattagccgttacagccagcggcaactgatggaaaccagccaccgccatttattgcaggcagaatcaggcgtctggctcaatatcgatggttatcatatgggagtgggcggggatgattcatggagcccgtcggtatcgccggaatttcaactgagcgcccggcattaccactaccagatcgcatggaaataaaatacctcattgggcagattgatcctgcccatttttaatgcaaggaaatccata

SEQ ID NO: 12 - DNA, 2520 bp, S. enterica subsp. IIIa
cggatgagcggcattttccgcgatgtttccctgctgcataagccgacgacgcagattacggatttccacctccatactcatctgaatgacgactttacccaggcggtgcttgaggcggaagttcgactggctggcggcgtgaacgacgatttgcagctcgtgcttcatctctggcagggagaaacgctggcgggagaggcccgcgccacgccgggaagtgaaattattgacgagcgcggcggctacgatgaccgcgccacgctacgcctcaatatcaatcgtccggcgctgtggagcgcggaaacgcctaatctgtatcgggcggtgatccaattgcgtaccgccgatggcgagctgattgaagcggaagcgtgtgacgttggcttccgccaggtccgcatcgataaggggctgttactgctaaatggtaaaccgctgctgatccgtgggaccaatcggcatgagcatcaccctgagcgcggccaggtgatggattacgatactatggtgcaggatattttgctgatgaagcagaataactttaatgccgtacgctgctcgcattaccccaatcatccgcagtggtacgccctatgtgaccgctatggtctgtacgtggtggatgaagccaacattgaaacccacggtatgacgccgatgaatcgcctgagcgatgacccggactggctacccgccatgagtcagcgtgtgacgcgcatggtgcagcgcgatcgcaaccatccgagcattatcatctggtcgttgggcaatgaatccgggcatggggccaaccatgatgcgctctaccgctggctgaaagcggaagatccttctcgcccggtacaatatgagggcggcggcgcggacaccgctgcgacggatattatctgcccgatgtatgcgcgcgttgatcaggatcagcccttcccggcagtgccaaagtggtcaatcaagaagtggctctcgctgcccggcgagcagcgaccgctcattctctgcgaatatgcgcatgcgatgggtaacagcttcggcggctttgccaaatactggcaggcgtttcgccagtatccgcgcttgcagggcggctttgtctgggactgggtagatcagtcgttgattaaatatgacgcggacggcaaaccgtggtctgcctacggcggagattttggcgacacgccaaatgatcgtcagttctgcatgaacgggctggtctttgccgatcgtacgccgcatccggcgttgtatgaagcgaaacacgtgcagcaatttttccagtttcgtttgctgccgggcgaagaacgccgcattgaagtacagagcgaatatctgttccgccacagtgataacgagatcctgcgctggatgctggcgcaggagggcaatcagttggccagtggtgaagttgtgctagatatcgccccgcaggggagacaaattatcctgctacctgcattcccgcagccggaaaccgcagggcagctttggctgacggtacgcgttgagcaaccgctggcgacaagctggtcggaggccgggcatatcagcgcctggcaacagtggccgttggaagaaaagctctgcgtatcgaaacccactcgcgccagcgttgcgcccgtcctgacgatgcgtgacggcgaattctgcgtcacgcaaggtaatttgcgttggcagttctgccgtcagcagggctggcttacacagttctggcgggatgatgaagcgcaattgctgaccccgcttatcgatcaattcacccgtgcgccgcttgataacgatattggcgtcagtgaagcgacacgcattgaccccaatgcctgggtggagcgttggaaggcggcaggacattactgcgcggagccagcgctgttgctgtgtgatgcagacgagctggcggatgcggtgctgataaccaccgcgcatgcctggcaatatcagggggcaacgctgtttatcagccgcaaaacctaccggattgatgatcatggcgaaatgcagatcgacatcggggtggaggttgccagtggaatgccgtacccggcgcggattggcttgagctgtcaacttgcgcaggtgaatgaaagggtggagtggctggggctggggccgcatgagaattatccggatcgcctgagcagcgcttgttttgatcgctggaatctcccgctggatgcgatgtatacgccgtatgtcttcccgactgaaaacggcctgcgttgcggtactcgccagttgcgttatggcgcgcaccagtggagcggcgattttcagttcaatattagccgctacagccagcggcaactgatggaaaccagccaccgtcatttattgcaggcagaagcaggcgtctggctcaatatcgatggttatcatatgggagtgggcggggatgattcatggagcccgtcggtatcgccggaatttcaactgagcgcccggcattaccactaccagatcgcatggaaataaaatacctcattgggcagattgatcctgcccatttttaatgcaaggaaatccata

SEQ ID NO: 13 - DNA, 1662 bp, S. enterica subsp. 1 serovar Typhimurium LT2
atgccacattttaatcctgttcctgtatcgaataaaaaattcgtctttgatgatttcatactcaacatggacggctccctgctacgctcagaaaagaaagtcaatattccgccaaaagaatatgccgttctggtcatcctgctcgaagccgccggcgagattgtgagtaaaaacaccttactggaccaggtatggggcgacgcggaagttaacgaagaatctcttacccgctgtatttatgccttacgacgtattctgtcggaagataaagagcatcgttacattgaaacactgtacggacagggctatcggtttaatcgtccggtcgtagtggtgtctccgccagcgccgcaacctacgactcatacattggcgatacttccttttcagatgcaggatcaggttcaatccgagagtctgcattactctatcgtgaagggattatcgcagtatgcgccctttggcctgagcgtgctgccggtgaccattacgaagaactgccgcagtgttaaggatattcttgagctcatggatcaattacgccccgattattatatctccgggcagatgatacccgatggtaatgataatattgtacagattgagatagttcgggttaaaggttatcacctgctgcaccaggaaagcattaagttgatagaacaccaacccgcttctctcttgcaaaacaaaattgcgaatcttttgctcagatgtattcccggacttcgctgggacacaaagcagattagcgagctaaattcgattgacagtactatggtttacttacgcggtaagcatgagttaaatcaatacaccccctatagcttacagcaagcgcttaaattgctgactcaatgcgttaacatgtcgccaaacagcattgcgccttactgtgcgctggcagaatgctacctcagcatggcgcaaatggggatttttgataaacaaaacgctatgatcaaagctaaagaacatgcgattaaggcgacagagctggaccacaataatccacaagctttaggattactggggctaattaatacgattcactcagaatacatcgtcgggagtttgctattcaaacaagctaacttactttcgcccatttctgcagatattaaatattattatggctggaatcttttcatggctggtcagttggaggaggccttacaaacgattaacgagtgtttaaaattggacccaacgcgcgcagccgcagggatcactaagctgtggattacctattatcataccggtattgatgatgctatacgtttaggcgatgaattacgctcacaacacctgcaggataatccaatattattaagtatgcaggttatgtttctttcgcttaaaggtaaacatgaactggcacgaaaattaactaaagaaatatccacgcaggaaataacaggacttattgctgttaatcttctttacgctgaatattgtcagaatagtgagcgtgccttaccgacgataagagaatttctggaaagtgaacagcgtatagataataatccgggattattaccgttagtgctggttgcccacggcgaagctattgccgagaaaatgtggaataaatttaaaaacgaagacaatatttggttcaaaagatggaaacaggatccccgcttgattaaattacggtaa

SEQ ID NO: 14 - DNA, 1662 bp, S. enterica subsp. 1 serovar Typhimurium
atgccacattttaatcctgttcctgtatcgaataaaaaattcgtctttgatgatttcatactcaacatggacggctccctcgtacgctcagaaaagaaagtcaatattccgccaaaagaatatgccgttctggtcatcctgctcgaagccgccggcgagattgtgagtaaaaacaccttactggaccaggtatggggcgacgcggaagttaacgaagaatctcttacccgctgtatttatgccttacgacgtattctgtcggaagataaagagcatcgttacattgaaacactgtacggacagggctatcggtttaatcgtccggtcgtagtggtgtctccgccagcgccgcaacctacgactcatacattggcgatacttccttttcagatgcaggatcaggttcaatccgagagtctgcattactctatcgtgaagggattatcgcagtatgcgccctttggcctgagcgtgctgccggtgaccattacgaagaactgccgcagtgttaaggatattcttgagctcatggatcaattacgccccgattattatatctccgggcagatgatacccgatggtaatgataatattgtacagattgagatagttcgggttaaaggttatcacctgctgcaccaggaaagcattaagttgatagaacaccaacccgcttctctcttgcaaaacaaaattgcgaatcttttgctcagatgtattcccggacttcgctgggacacaaagcagattagcgagctaaattcgattgacagtactatggtttacttacgcggtaagcatgagttaaatcaatacaccccctatagcttacagcaagcgcttaaattgctgactcaatgcgttaacatgtcgccaaacagcattgcgccttactgtgcgctggcagaatgctacctcagcatggcgcaaatggggatttttgataaacaaaacgctatgatcaaagctaaagaacatgcgattaaggcgacagagctggaccacaataatccacaagctttaggattactggggctaattaatacgattcactcagaatacatcgtcgggagtttgctattcaaacaagctaacttactttcgcccatttctgcagatattaaatattattatggctggaatcttttcatggctggtcagttggaggaggccttacaaacgattaacgagtgtttaaaattggacccaacgcgcgcagccgcagggatcactaagctgtggattacctattatcataccggtattgatgatgctatacgtttaggcgatgaattacgctcacaacacctgcaggataatccaatattattaagtatgcaggttatgtttctttcgcttaaaggtaaacatgaactggcacgaaaattaactaaagaaatatccacgcaggaaataacaggacttattgctgttaatcttctttacgctgaatattgtcagaatagtgagcgtgccttaccgacgataagagaatttctggaaagtgaacagcgtatagataataatccgggattattaccgttagtgctggttgcccacggcgaagctattgccgagaaaatgtggaataaatttaaaaacgaagacaatatttggttcaaaagatggaaacaggatccccgcttgattaaattacggtaa

SEQ ID NO: 15 - DNA, 24 bp, Artificial sequence
  hilA target nucleotide sequence for forward primer
tcgcatatctattattaggcccta

SEQ ID NO: 16 - DNA, 24 bp, Artificial sequence
  hilA target nucleotide sequence for forward primer
tcgcataactattattaggcccta

SEQ ID NO: 17 - DNA, 23 bp, Artificial sequence
  hilA target nucleotide sequence for reverse primer
taaggtgtaaaagagccgttatc

SEQ ID NO: 18 - DNA, 23 bp, Artificial sequence
  hilA target nucleotide sequence for reverse primer
caaggtgtaaaagagccgttatc

SEQ ID NO: 19 - DNA, 24 bp, Artificial sequence
  hilA forward primer
agcgtatagataataatccgggat

SEQ ID NO: 20 - DNA, 24 bp, Artificial sequence
  hilA forward primer
agcgtattgataataatccgggat

SEQ ID NO: 21 - DNA, 23 bp, Artificial sequence
  hilA reverse primer
attccacattttctcggcaatag

SEQ ID NO: 22 - DNA, 23 bp, Artificial sequence
  hilA reverse primer
gttccacattttctcggcaatag

SEQ ID NO: 23 - DNA, 24 bp, Artificial sequence
  hilA target nucleotide sequence for probe
gtgggcaaccagcactaacggtaa

SEQ ID NO: 24 - DNA, 24 bp, Artificial sequence
  hilA probe
ttaccgttagtgctggttgcccac

(19) United States
(12) Patent Application Publication
(10) Pub. No.: US 2011/0244453 A1
(43) Pub. Date: Oct. 6, 2011

(54) SALMONELLA DETECTION ASSAY

(75) Inventors: Katie Hopkins, London (GB); Andy Lawson, London (GB)

(73) Assignee: HEALTH PROTECTION AGENCY, London (GB)

(21) Appl. No.: 12/995,428

(22) PCT Filed: Jun. 3, 2009
(86) PCT No.: PCT/GB2009/050617

§ 371 Date: Jun. 13, 2011

(30) Foreign Application Priority Data
Jun. 3, 2008 (GB) 0810090.1
Mar. 13, 2009 (GB) 0904364.7

(51) Int. Cl. C12Q 1/68; C07H 21/00

(52) U.S. Cl. 435/6.11; 435/6.12; 536/24.33; 536/24.32

(57) ABSTRACT

There is provided a method and reagents for detecting S. enterica subsp. IIIa and/or IIIb in a sample, the method comprising: (a) contacting the sample with a pair of forward and reverse oligonucleotide primers, wherein said forward and reverse primers hybridise to target nucleic acid sequences located within the lacZ gene of S. enterica subsp. III, or the complement thereof; (b) extending said forward and reverse primers to generate an amplification product; and (c) detecting the amplification product. There is also provided a method a reagents for detecting S. enterica subsp. I in a sample, the method comprising: (a) contacting the sample with a pair of forward and reverse oligonucleotide primers, wherein said forward and reverse primers hybridise to target nucleic acid sequences located within the hilA gene of S. enterica subsp. I, or the complement thereof; (b) extending said forward and reverse primers to generate an amplification product; and (c) detecting the amplification product.

The present invention relates to an assay for detecting Salmonella sp. in a sample, and to reagents and kits therefor.

The family Enterobacteriaceae includes coliform bacteria (genera such as Enterobacter, Escherichia, Hafnia, Klebsiella and Serratia) and enteric bacteria such as Salmonella and Shigella.

Salmonella bacteria cause food poisoning, typhoid fever and paratyphoid fever.

Transmission occurs by eating contaminated food, mainly of animal origin, or by faecal contamination from an infected person or animal. The incubation period is 12 to 72 hours. Secondary cases are common in outbreaks. Food handlers who practice good hygiene are very rarely responsible for initiating outbreaks. On average 13,000 cases are reported in the UK each year—however, this may be a significant underestimate of actual cases.

Salmonella enterica bacteria are divided into seven groups or subspecies (subsp.), namely: S. enterica subsp. enterica (subsp. I), salamae (subsp. II), arizonae and diarizonae (subsp. IIIa and IIIb respectively), houtenae (subsp. IV), bongori (subsp. V—now considered a separate species), indica (subsp. VI) and as yet unnamed subsp. VII. Within these subspecies more than 2500 different serotypes of salmonella have been identified and serotyping remains the principal method of epidemiologically sub-typing these bacteria.

Salmonella enterica of subsp. I are by far the most important human pathogens, accounting for the vast majority of human cases and >95% of the isolates received in the laboratory in the UK. Subsp. IIIa and IIIb (arizonae and diarizonae) are the next most frequent, accounting for 2 to 3% of isolates in the UK, with the remaining subspecies being very rare.

Despite the comparative rarity of Salmonella enterica other than subsp. I, accurate subspecies identification is epidemiologically important because a number of identical serotypes occur in different subspecies (i.e. it is possible for isolates of subsp. I and subsp. III to appear identical by serotyping alone).

S. enterica subspecies IIIa (arizonae) and IIIb (diarizonae) are naturally found in reptiles and have been responsible for outbreaks in turkeys and sheep. The organisms can also be transmitted to humans via direct contact with reptiles or ingestion of snake meat products, usually resulting in gastroenteritis but also leading to bacteraemia, sepsis, osteomyelitis and meningitis. Human infections are relatively uncommon; cases of severe infection tend to occur in patients with impaired immunity or in young children, and can be difficult to eradicate. Exotic reptiles are increasingly popular as pets, leading to a concurrent increase in human infections due to uncommon Salmonella isolates including subspecies arizonae and diarizonae.

Many different conventional culture media and enrichment regimes have been proposed for Salmonella species, which allow detection in typically 18 to 48 hours. S. enterica subspecies I is unable to ferment lactose and this property is the basis of many selective isolation media used for Salmonella. However, a significant proportion of S. enterica subspecies III strains ferment lactose and would not be detected using these conventional techniques.

Identification of S. enterica subspecies arizonae and diarizonae with routine biochemistry and serology can be problematic and may take between 14 and 28 days for a definitive result.

In this regard, several conventional biochemical tests are employed for differentiating S. enterica subspecies arizonae and diarizonae from the other subspecies of S. enterica. These include dulcitol and lactose fermentation, malonate utilisation, and hydrolysis of gelatine and ONPG (o-nitrophenyl-β-D-galactopyranoside). The antigenic structure of these subspecies is often difficult to determine; strains with the biochemical reactions of these subspecies usually need to be sent to a specialist laboratory for full identification.

Known molecular detection assays for Salmonella used by the food industry detect the Salmonella genus as a whole (e.g. based on detecting InvA (inv=invasion gene) or ttr (ttr=tetrationate respiration)), and do not therefore distinguish all of the salmonella subspecies, including Salmonella bongori.

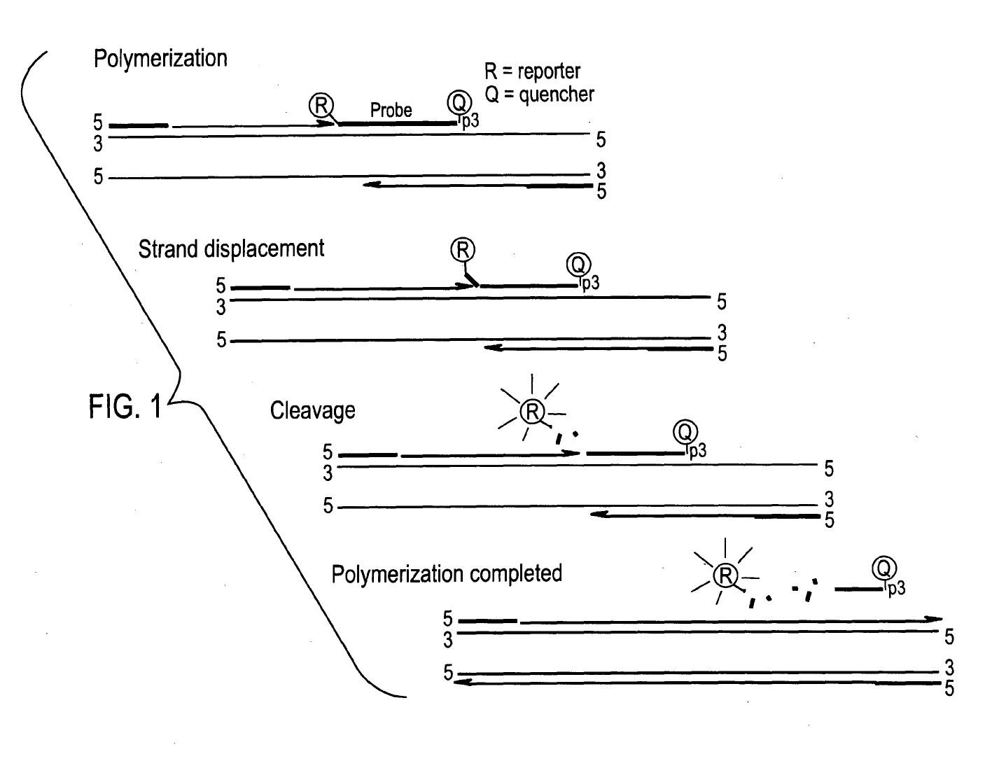

A recent development of the standard PCR assay is the emergence of real-time detection methods such as the Applied Biosystems Taqman assay, which employs a sequence-specific fluorescently labelled probe (see FIG. 1).

During the Taqman reaction, the primers and probe bind to the target sequence, if present. As the primers are extended, the bound probe obstructs the progress of one of the extending strands. This obstruction is then circumvented by Taq polymerase, which possesses a 5′ exonuclease activity and enzymatically degrades the single-stranded oligonucleotide probe. As the probe is cleaved the two fluorophores present on the probe are separated, thus altering the relative fluorescent signal. On each successive round of PCR thermal cycling the target nucleotide sequence accumulates, and for every DNA molecule synthesised a probe will be cleaved. The resulting fluorescent signal is cumulative and increases exponentially during PCR amplification.

There is a need in the art for an improved assay for detecting Salmonella species (such as S. enterica, for example subsp. IIIa and/or IIIb) in a sample.

The present invention meets this need by providing methods for detecting a Salmonella subsp. in a sample. In one embodiment, said method is based on detection of a nucleic acid sequence (such as a gene sequence) that is specific to that Salmonella subsp.

Thus, in one embodiment, the invention provides a method for detecting a Salmonella subsp. in a sample, the method comprising: (a) contacting the sample with a pair of forward and reverse oligonucleotide primers, wherein said forward and reverse primers hybridise to target nucleic acid sequences located within a nucleic acid sequence that is specific to that Salmonella subsp., or the complement thereof; (b) extending said forward and reverse primers to generate an amplification product; and (c) detecting the amplification product. In one embodiment, the amplification product is detected by a method comprising contacting the sample with an oligonucleotide probe that forms a hybridisation complex with the amplification product, if present; and detecting the hybridisation complex.

In one, embodiment, the present invention provides a method for detecting S. enterica subsp. IIIa and/or IIIb in a sample, the method comprising:

(a) contacting the sample with a pair of forward and reverse oligonucleotide primers, wherein said forward and reverse primers hybridise to target nucleic acid sequences located within the lacZ gene of S. enterica subsp. III, or the complement thereof;

(b) extending said forward and reverse primers to generate an amplification product; and

(c) detecting the amplification product.

The method advantageously provides a highly sensitive, specific, rapid and robust molecular diagnostic assay for Salmonella enterica subsp. IIIa (arizonae) and/or IIIb (diarizonae), which has the potential to replace the existing laborious and long turnaround biochemical assays presently being used.

In one embodiment illustrated in the Examples, the assay shows 100% specificity and 99% sensitivity. In one embodiment, the assay does not detect any Hafnia, Citrobacter, Enterobacter, Escherichia or Proteus spp. In one embodiment, the assay identifies Salmonella enterica subsp. IIIa and/or IIIb in less than 2 hours, compared to an average minimum turnaround time of 14-28 days for full routine biochemistry and serology-based identification methods.

In one embodiment, the assay is useful for screening clinical, veterinary, food or research-based samples for S. enterica subsp. IIIa and/or IIIb, to aid monitoring, or to monitor epidemiology and natural history of the disease, along with other potential research.

To the best of our knowledge, the lacZ gene of S. enterica subsp. IIIa and/or IIIb has not previously been used as a target for detecting Salmonella .

Three versions of the sequence for the lacZ gene of S. enterica subsp. IIIa and IIIb are publically available, and are represented herein by SEQ ID NOs: 1, 11 and 12. SEQ ID NO: 1 corresponds to the lacZ gene of S. enterica subsp. diarizonae (IIIb) publically available under Accession number AY746956. SEQ ID NO: 11 corresponds to the lacZ gene of S. enterica subsp. diarizonae (IIIb) provided by the University of Washington. SEQ ID NO: 12 corresponds to the lacZ gene of S. enterica subsp. arizonae (IIIa) provided by the University of Washington. SEQ ID NOs: 1 and 11 share approximately 99.9% identity over their entire length. SEQ ID NO: 12 shares over 98.5% identity with SEQ ID NOs: 1 and 11 over their entire length.

Thus, in one embodiment, the lacZ gene of S. enterica subsp. III comprises (or consists of) a nucleotide sequence having at least 90% identity (such as at least 91, 92, 93, 94, 95, 96, 97, 98, 99 or 100% identity) to a nucleotide sequence selected from SEQ ID NOs: 1, 11 or 12.

In one embodiment, the method comprises contacting the sample with a pair of forward and reverse oligonucleotide primers, wherein said forward and reverse primers hybridise to target nucleic acid sequences located within a nucleotide sequence having at least 90% identity (such as at least 91, 92, 93, 94, 95, 96, 97, 98, 99 or 100% identity) to a lacZ nucleotide sequence selected from SEQ ID NOs: 1, 11 or 12.

All S. enterica subspecies III (arizonae and disarizonae) have the lacZ gene. The lacZ gene encodes a β-galactosidase, an enzyme which breaks the β1-4 glycosidic links found in several carbohydrates (such as that between the galactose and glucose monosaccharides that constitute the lactose molecule).

The Applicant has unexpectedly identified a region of the lacZ gene that is specific to S. enterica subsp. IIIa and IIIb, and conserved between strains of S. enterica subsp. IIIa and IIIb.

In one embodiment, the method comprises amplification and detection of a region of the lacZ gene that is conserved amongst strains of S. enterica subsp. IIIa and IIIb.

In one embodiment, the method comprises amplification and detection of a region of the lacZ gene that is distinct from the lacZ gene of all other Enterobacteriaceae.

Some S. enterica subsp. III (approx. 25% arizonae and approx. 75% disarizonae) are able to ferment lactose and these, presumably, also have the lacY gene, which encodes a β-galactoside permease responsible for transporting lactose from outside the bacterial cell to the inside where the LacZ exerts its effect.

Almost without exception, all S. enterica subspecies I lack both lacZ and lacY and are thus unable to ferment lactose.

The presence of an active β-galactosidase (and hence the lacZ gene in Salmonella) can be detected phenotypically by the ortho-nitrophenyl-β-galactoside (ONPG) test. ONPG is a sugar like molecule that contains a β1-4 glycosidic link; in the presence of β-galactosidase this link is cleaved, producing orthonitrophenol, an intensely yellow compound that is a visible indicator of positive activity.

Throughout this application, the term “S. enterica subsp. III” embraces both subsp. IIIa and/or IIIb.

Throughout this application, the term “S. enterica subsp. IIIa” is equivalent to the term “S. enterica subsp. arizonae”, and the term “S. enterica subsp. IIIb” is equivalent to the term “S. enterica subsp. diarizonae”.

In one embodiment, the sample is (or is derived from) a clinical, veterinary, food, water, environmental or faecal sample or bacterial culture or DNA extract.

In one embodiment, the target nucleic acid sequence to which the forward primer hybridises is specific to S. enterica subsp. III. In one embodiment, the target nucleic acid sequence to which the reverse primer hybridises is specific to S. enterica subsp. III.

In one embodiment, extension of the forward and reverse primers generates an amplification product comprising a nucleic acid sequence that is specific to S. enterica subsp. III.

In general, a reverse primer is designed to hybridise to a target nucleic acid sequence within the coding (sense) strand of a target nucleic acid, and a forward primer is designed to hybridise to a target nucleic acid sequence within the complementary (i.e. anti-sense) strand of the target nucleic acid.

The term “complement of a nucleic acid sequence” refers to a nucleic acid sequence having a complementary nucleotide sequence as compared to a reference nucleotide sequence.

In one embodiment, the forward primer hybridises to a target nucleic acid sequence (a ‘forward primer target sequence’) located within the complement of SEQ ID NO: 1, 11 or 12.

In one embodiment, the forward primer target sequence has a length in the range of 10-40 consecutive nucleotides of the complement of SEQ ID NO: 1, 11 or 12, such as at least 11, 12, 13, 14, 15, 16, 17, 18, 19 or 20 consecutive nucleotides of the complement of SEQ ID NO: 1, 11 or 12, such as up to 38, 35, 32, 30, 29, 28, 27, 26, 25, 24, 23, 22, 21 or 20 consecutive nucleotides of the complement of SEQ ID NOs: 1, 11 or 12. For example, the reverse primer target sequence may have a length of 15-25 consecutive nucleotides of the complement of SEQ ID NO: 1, 11 or 12, such as a length of about 20 consecutive nucleotides of the complement of SEQ ID NO: 1, 11 or 12.

In one embodiment, the forward primer target sequence is specific to S. enterica subsp. IIIa and/or IIIb.

In one embodiment, the forward primer hybridises to a target sequence located between residues 1-2050 of the complement of SEQ ID NO: 1, 11 or 12. Within this range, the forward primer target sequence may be located in a region from residue 200, 400, 600, 800, 1000, 1100, 1200, 1300, 1400, 1500, 1600, 1700, 1800, 1900, 1910, 1920, 1930, 1940, 1945, 1950, 1951, 1952, 1953, 1954, 1955, 1956, 1957 or 1958 of the complement of SEQ ID NO: 1, 11 or 12. Within this range, the forward primer target sequence may be located in a region up to residue 2040, 2030, 2020, 2010, 2000, 1990, 1985, 1984, 1983, 1982, 1981, 1980, 1979, 1978 or 1977 of the complement of SEQ ID NO: 1, 11 or 12. For example, the forward primer may hybridise to a target sequence located between residues 1930-2000, such as between residues 1950-1980 of the complement of SEQ ID NO: 1, 11 or 12. In one embodiment, the forward primer target sequence is defined by residues 1958-1977 of the complement of SEQ ID NO: 1, 11 or 12.

For the avoidance of doubt, the above numbering system applied to the nucleic acid residues of the complementary strand of SEQ ID NOs: 1, 11 or 12 is based on the numbering of the nucleic acids of SEQ ID NOs: 1, 11 or 12 to which they are complementary.

In one embodiment, the forward primer hybridises to a target nucleic acid sequence that comprises (or consists of) SEQ ID NO: 2 (shown below) or a nucleotide sequence that is at least 75% identical thereto (such as 80, 85, 90, 91, 92, 93, 94, 95, 96, 97, 98, 99 or 100% identical thereto), or a fragment thereof.

| Forward primer | |
|---|---|
| target SEQ ID NO: | Sequence |
| SEQ ID NO: 2 | CGT TTT GGA TGG CCT AAC TA |

In one embodiment, a fragment of SEQ ID NO: 2 (or sequence variants thereof as defined above), comprises (or consists of) at least 15 consecutive nucleotides thereof, such as at least 16, 17, 18 or 19 consecutive nucleotides thereof.

In one embodiment, the reverse primer hybridises to a target nucleic acid sequence (a ‘reverse primer target sequence’) located within SEQ ID NO: 1, 11 or 12.

In one embodiment, the reverse primer target sequence has a length in the range of 10-40 consecutive nucleotides of SEQ ID NO: 1, 11 or 12, such as at least 11, 12, 13, 14, 15, 16, 17, 18, 19 or 20 consecutive nucleotides of SEQ ID NO: 1, 11 or 12, such as up to 38, 35, 32, 30, 29, 28, 27, 26, 25, 24, 23, 22, 21 or 20 consecutive nucleotides of SEQ ID NO: 1, 11 or 12. For example, the reverse primer target sequence may have a length of 15-25 consecutive nucleotides of SEQ ID NO: 1, 11 or 12, such as a length of about 20 consecutive nucleotides of SEQ ID NO: 1, 11 or 12.

In one embodiment, the reverse primer target sequence is specific to S. enterica subsp. IIIa and/or IIIb.

In one embodiment, the reverse primer hybridises to a target sequence located between residues 2050-2520 of SEQ ID NO: 1, 11 or 12, such as a target sequence located in a region from residue 2055, 2060, 2061, 2062, 2063, 2064, 2065, 2066, 2067 or 2068 of SEQ ID NO: 1, 11 or 12, such as a target sequence located in a region up to residue 2500, 2400, 2300, 2200, 2150, 2140, 2130, 2120, 2110, 2100, 2095, 2094, 2093, 2092, 2091, 2090, 2089, 2088 or 2087 of SEQ ID NO: 1, 11 or 12. For example, the reverse primer may hybridise to a target sequence located between residues 2050-2110, such as residues 2060-2095 of SEQ ID NO: 1, 11 or 12. In one embodiment, the reverse primer target sequence is defined by residues 2068-2087 of SEQ ID NO: 1, 11 or 12.

In one embodiment, the reverse primer hybridises to a target nucleic acid sequence that comprises (or consists of) a nucleotide sequence that is at least 75% identical to (such as 80, 85, 90, 91, 92, 93, 94, 95, 96, 97, 98, 99 or 100% identical to) a nucleotide sequence of SEQ ID NO: 3 (shown below), or a fragment thereof.

| Reverse primer | |
|---|---|
| target SEQ ID NO: | Sequence |
| SEQ ID NO: 3 | AGG TGA ATG AAA GGG TGG AG |

In one embodiment, a fragment of SEQ ID NO: 3 (or sequence variants thereof as defined above), comprises (or consists of) at least 15, 16, 17, 18 or 19 consecutive nucleotides thereof.

In one embodiment, the forward primer is 15-30 nucleotides long, such as at least 16, 17, 18, 19 or 20 nucleotides long, such as up to 29, 28, 27, 26, 25, 24, 23, 22, 21 or 20 nucleotides long. For example, the reverse primer may be 18-22 nucleotides long, such as about 20 nucleotides long.

In one embodiment, the reverse primer is 15-30 nucleotides long, such as at least 16, 17, 18, 19 or 20 nucleotides long, such as up to 29, 28, 27, 26, 25, 24, 23, 22, 21 or 20 nucleotides long. For example, the reverse primer may be 18-22 nucleotides long, such as about 20 nucleotides long.

In one embodiment, the forward primer comprises (or consists of) a nucleotide sequence having at least 75% identity to (such as at least 80, 85, 90, 91, 92, 93, 94, 95, 96, 97, 98, 99 or 100% identity to) a nucleotide sequence of SEQ ID NO: 4 (shown below). Conservative substitutions may be useful in this regard.

| Forward Primer | |
|---|---|
| SEQ ID NO: | Sequence |
| SEQ ID NO: 4 | GCA AAA CCT ACC GGA TTG AT |

Variants of SEQ ID NO: 4 may alternatively be defined by reciting the number of nucleotides that differ between the variant sequences and SEQ ID NO: 4. Thus, in one embodiment, the forward primer may comprise (or consist of) a nucleotide sequence that differs from SEQ ID NO: 4 at no more than 5 nucleotide positions, for example at no more than 4, 3, 2 or 1 nucleotide positions. Conservative substitutions may be useful in this regard.

Fragments of the above-mentioned forward primer sequence (and sequence variants thereof as defined above) may also be employed. In one embodiment, the forward primer may comprise (or consist of) a fragment of SEQ ID NO: 4 (and sequence variants thereof as defined above), wherein said fragment comprises (or consists of) at least 15 consecutive nucleotides thereof, such as at least 16, 17, 18 or 19 consecutive nucleotides thereof.

In one embodiment, the reverse primer comprises (or consists of) a nucleotide sequence having at least 75% identity to (such as at least 80, 85, 90, 91, 92, 93, 94, 95, 96, 97, 98, 99 or 100% identity to) a nucleotide sequence of SEQ ID NO: 5. Conservative substitutions may be useful in this regard.

| Reverse Primer | |
|---|---|
| SEQ ID NO: | Sequence |
| SEQ ID NO: 5 | TCC ACT TAC TTT CCC ACC TC |

Variants of SEQ ID NO: 5 may alternatively be defined by reciting the number of nucleotides that differ between the variant sequences and SEQ ID NO: 5. In one embodiment, the reverse primer may comprise (or consist of) a nucleotide sequence that differs from SEQ ID NO: 5 at no more than 5 nucleotide positions, for example at no more than 4, 3, 2 or 1 nucleotide positions. In this regard, conservative substitutions may be useful.

Fragments of the above-mentioned reverse primer sequences (and sequence variants thereof as defined above) may also be employed. In one embodiment, the reverse primer may comprise (or consist of) a fragment of SEQ ID NO: 5 (and sequence variants thereof as defined above), wherein said fragment comprises (or consists of) at least 15 consecutive nucleotides thereof, such as at least 16, 17, 18 or 19 consecutive nucleotides thereof.

The forward and reverse primers of the present invention are designed to bind to the target nucleic acid sequence based on the selection of desired parameters, using conventional software, such as Primer Express (Applied Biosystems).

The term ‘hybridises’ is equivalent and interchangeable with the term ‘binds’.

In one embodiment, the forward primer is sequence-specific and hybridises specifically to the forward primer target nucleic acid sequence within SEQ ID NO: 1, 11 or 12.

In one embodiment, the reverse primer is sequence-specific and hybridises specifically to the reverse primer target nucleic acid sequence within SEQ ID NO: 1, 11 or 12.

In one embodiment, the binding conditions are such that a high level of specificity is provided. In one embodiment, the melting temperature (Tm) of the forward and reverse primers is in excess of 68° C., such as about 72° C.

In one embodiment, the forward primer and/or the reverse primer comprises a tag or label. In one embodiment, said tag or label is incorporated into the amplification product when the primer is extended. The tag or label may be located at the 5′ or 3′ end of the forward and/or reverse primer, for example at the 5′ end of the reverse primer.

Examples of suitable labels include detectable labels such as radiolabels or fluorescent or coloured molecules. By way of example, the label may be digoxygenin, fluorescein-isothiocyanate (FITC) or R-phycoerythrin. The label may be a reporter molecule, which is detected directly, such as by exposure to photographic or X-ray film. Alternatively, the label is not directly detectable, but may be detected indirectly, for example, in a two-phase system. An example of indirect label detection is binding of an antibody to the label.

Examples of suitable tags include biotin and streptavidin. Other exemplary tags include receptors, ligands, antibodies, antigens, haptens and epitopes.

Amplification may be carried out using methods and platforms known in the art, for example PCR, such as real-time PCR. In one embodiment, amplification is carried out using a real-time Taqman® PCR platform.

In one embodiment, amplification can be carried using any amplification platform—as such, an advantage of this embodiment of the assay is that it is platform independent and not tied to any particular instrument.

In the presence of a suitable polymerase and DNA precursors (dATP, dCTP, dGTP and dTTP), the forward and reverse primers are extended in a 5′ to 3′ direction, thereby initiating the synthesis of new nucleic acid strands that are complementary to the individual strands of the target nucleic acid. The primers thereby drive amplification of the target S. enterica subsp. IIIa and/or IIIb nucleic acid, thereby generating an amplification product comprising said target S. enterica subsp. IIIa and/or IIIb nucleic acid sequence. A skilled person would be able to determine suitable conditions for promoting amplification.

In this application, the expressions “amplification product”, “amplified nucleic acid sequence” and “amplicon” are used interchangeably and have the same meaning.

In one embodiment, the amplification product is in the range of 50-250 nucleotides, for example at least 60, 70, 80, 90, 95, 100, 105, 110, 115, 120 or 125 nucleotides, for example up to 225, 200, 190, 180, 170, 160, 150, 145, 140 or 135 nucleotides. In one embodiment, the amplification product is in the range of 110-150 nucleotides, such as in the range of 125-135 nucleotides, such as about 130 nucleotides.

The detection step may be carried out by any known means.

In one aspect, the amplification product is tagged or labelled, and the detection method comprises detecting the tag or label. In one embodiment, the tag or label is incorporated into the amplification product during the amplification step. In one embodiment, the forward and/or reverse primer comprises a tag or label, and the tag or label is incorporated into the amplification product when the primer is extended during the amplification step. The tag or label may be located at the 5′ or 3′ end of the forward or reverse primer, for example at the 5′ end of the reverse primer.

Thus, in one embodiment, the amplification product is labelled, and the assay comprises detecting the label (e.g. following removal of primer) and correlating presence of label with presence of amplification product, and hence the presence of S. enterica subsp. IIIa and/or IIIb. The label may comprise a detectable label such as a radiolabel or a fluorescent or coloured molecule. By way of example, the label may be digoxygenin, fluorescein-isothiocyanate (FITC) or R-phycoerythrin. The label may be a reporter molecule, which is detected directly, such as by exposure to photographic or X-ray film. Alternatively, the label is not directly detectable, but may be detected indirectly, for example, in a two-phase system. An example of indirect label detection is binding of an antibody to the label.

In one embodiment, the amplification product is tagged, and the assay comprises capturing the tag (e.g. following removal of primer) and correlating presence of the tag with presence of amplification product, and hence the presence of target S. enterica subsp. IIIa and/or IIIb. In one embodiment, the tag is captured using a capture molecule, which may be attached (e.g. coated) onto a substrate or solid support, such as a membrane or magnetic bead.

Capture methods employing magnetic beads are advantageous because the beads (plus captured, tagged amplification product) can easily be concentrated and separated from the sample, using conventional techniques known in the art.

Examples of suitable tags include “complement/anti-complement pairs”. The term “complement/anti-complement pair” denotes non-identical moieties that form a non-covalently associated, stable pair under appropriate conditions. For instance, biotin and avidin (or streptavidin) are members of a complement/anti-complement pair. Other exemplary complement/anti-complement pairs include receptor/ligand pairs, antibody/antigen (or hapten or epitope) pairs, and the like. Where subsequent dissociation of the complement/anti-complement pair is desirable, the complement/anti-complement pair may have a binding affinity of less than 10⁹ M⁻¹.

In one embodiment, the tag is selected from biotin and streptavidin. In this regard, a biotin tag may be captured using streptavidin, which may be coated onto a substrate or support such as a bead (for example a magnetic bead) or membrane. Likewise, a streptavidin tag may be captured using biotin, which may be coated onto a substrate or support such as a bead (for example a magnetic bead) or membrane. Other exemplary pairs of tags and capture molecules include receptor/ligand pairs and antibody/antigen (or hapten or epitope) pairs.

Thus, in one embodiment, the amplification product incorporates a biotin tag, and the detection step comprises contacting the sample with a streptavidin-coated magnetic bead, which captures the biotin-tagged amplification product. The magnetic bead (plus captured, tagged amplification product) can then be separated from the sample, thereby separating the amplification product from the sample. The amplification product can then be detected by any known means.

In one embodiment, the nucleic acid sequence of the amplification product is determined. Sequencing of the amplification product may be carried out by any known means. For example (after melting off the unlabelled strand of DNA with sodium hydroxide), a colorimetric sequencing system may be employed, such as the Trimgen Mutector™ detection system.

In one aspect, the amplification product is detected by a method comprising contacting the sample with an oligonucleotide probe under conditions allowing the formation of hybridisation complexes between the probe and the amplification product, and detecting the hybridisation complexes. In one embodiment, the probe is specific for the amplification product.

In one embodiment, the probe is 15-40 nucleotides long, for example at least 16, 17, 18, 19, 20, 21, 22 or 23 nucleotides long, for example up to 39, 38, 37, 36, 35, 34, 33, 32, 31, 30, 29, 28, 27, 26 or 25 nucleotides long. In one embodiment, the probe is 20-30 nucleotides long, such as 22-26 nucleotides long. In one embodiment, the probe is about 24 nucleotides long.

In one embodiment, the target nucleotide sequence to which the probe hybridises within the amplification product is 15-40 nucleotides long, such as at least 16, 17, 18, 19, 20, 21, 22 or 23 nucleotides long, such as up to 39, 38, 37, 36, 35, 34, 33, 32, 31, 30, 29, 28, 27, 26 or 25 nucleotides long. For example, the target nucleotide sequence for the probe may be 20-30 nucleotides long, such as 22-26 nucleotides long. In one embodiment, the probe binds a target nucleotide sequence that is about 24 nucleotides long.

Probes are designed to hybridise to their target sequence within the amplification product based on a selection of desired parameters, using conventional software. The binding conditions may be such that a high level of specificity is provided—i.e. hybridisation of the probe to the amplification product occurs under “stringent conditions”. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridises to a perfectly matched probe. In one embodiment, the Tₘ of probes of the present invention, at a salt concentration of about 0.02M or less at pH 7, is above 60° C., such as about 70° C.

Premixed binding solutions are available (e.g. EXPRESSHYB Hybridisation Solution from CLONTECH Laboratories, Inc.), and hybridisation can be performed according to the manufacturer's instructions. Alternatively, a person skilled in the art can devise suitable variations of these binding conditions.

Probes can be screened to minimise self-complementarity and dimer formation (probe-probe binding). Probes of the present invention may be selected so as to have minimal homology with human DNA. The selection process may involve comparing a candidate probe sequence with human DNA and rejecting the probe if the homology is greater than 50%. The aim of this selection process is to reduce annealing of probe to contaminating human DNA sequences and hence allow improved specificity of the assay.

In one embodiment, the target binding sequence for the probe is located within the complement of SEQ ID NO: 1, 11 or 12. In one embodiment, the probe binds a target nucleotide sequence located between residues 1900-2100 of the complement of SEQ ID NO: 1, 11 or 12. Within this range, the probe target sequence may be located from residue 1910, 1920, 1930, 1940, 1950, 1960, 1965, 1970, 1975, 1976, 1977, 1978, 1979, 1980 or 1981 of the complement of SEQ ID NO: 1, 11 or 12. Within this range, the probe target sequence may be located up to residue 2090, 2080, 2070, 2060, 2050, 2040, 2030, 2020, 2015, 2010, 2009, 2008, 2007, 2006, 2005 or 2004 of the complement of SEQ ID NO: 1, 11 or 12. For example, the forward primer may hybridise to a target sequence located between residues 1960-2020, such as residues 1975-2010 of the complement of SEQ ID NO: 1, 11 or 12. In one embodiment, the forward primer target sequence is defined by residues 1981-2004 of the complement of SEQ ID NO: 1, 11 or 12.

In one embodiment, the target binding sequence for the probe comprises (or consists of) a nucleotide sequence that is at least 75% identical (such as at least 80, 85, 90, 91, 92, 93, 94, 95, 96, 97, 98, 99 or 100% identical) to a nucleotide sequence of SEQ ID NO: 6 (shown below), or to a fragment thereof having at least 18 consecutive nucleotides thereof (such as at least 19, 20, 21, 22 or 23 consecutive nucleotides thereof).

| Probe target | |
|---|---|
| SEQ ID NO: | Sequence |
| SEQ ID NO: 6 | GTACCGCTTTACGTCTAGCTGTAG |

In one aspect, the oligonucleotide probe comprises (and may consist of) a nucleotide sequence having at least 75% identity (such as at least 80, 85, 90, 91, 92, 93, 94, 95, 96, 97, 98, 99 or 100% identity) to a nucleotide sequence of SEQ ID NO: 7 (shown below). In this regard, conservative substitutions may be useful.

| Probe SEQ ID NO: | Sequence |
|---|---|
| SEQ ID NO: 7 | CATGGCGAAATGCAGATCGACATC |

An alternative means for defining variant probe sequences is by defining the number of nucleotides that differ between the variant sequence and the reference probe sequence.

Thus, in one embodiment, a probe of the present invention comprises (or consists of) a nucleic acid sequence that differs from SEQ ID NO: 7 by no more than 6 nucleotides, for example by no more than 5, 4, 3, 2 or 1 nucleotides. In this regard, conservative substitutions may be useful.

A fragment of the above-mentioned probe sequence may also be employed, wherein the fragment comprises at least 18 consecutive nucleotides of SEQ ID NO: 7. Thus, in one embodiment, a probe of the present invention comprises (or consists of) a fragment of SEQ ID NO: 7 (or sequence variants thereof as defined above), wherein said fragment comprises at least 18, 19, 20, 21, 22 or 23 consecutive nucleotides thereof.

Following binding, washing under stringent (e.g. highly stringent) conditions removes unbound oligonucleotides. Typical stringent washing conditions include washing in a solution of 0.5-2×SSC with 0.1% SDS at 55-65° C. Typical highly stringent washing conditions include washing in a solution of 0.1-0.2×SSC with 0.1% SDS at 55-65° C. A skilled person can readily devise equivalent conditions—for example, by substituting SSPE for the SSC in the wash solution.

In one embodiment, the probe comprises a label. Thus, in one embodiment, following hybridisation of labelled probe to amplification product, the label is associated with the bound amplification product. Thus, in one embodiment, the assay comprises detecting the label (e.g. following separation of unbound probe from the sample) and correlating presence of label with presence of probe bound to amplification product, and hence the presence of S. enterica subsp. IIIa and/or IIIb.

The label may comprise a detectable label such as a radiolabel, fluorescent molecule, enzymatic marker or chromogenic marker—e.g. a dye that produces a visible colour change upon hybridisation of the probe. By way of example, the label may be digoxygenin, fluorescein-isothiocyanate (FITC) or R-phycoerythrin. The label may be a reporter molecule, which is detected directly, such as by exposure to photographic or X-ray film. Alternatively, the label is not directly detectable, but may be detected indirectly, for example, in a two-phase system. An example of indirect label detection is binding of an antibody to the label.

In one embodiment, the probe comprises a tag. Hence, following hybridisation of tagged probe to amplification product, the tag is associated with the bound amplification product. Thus, in one embodiment, the assay comprises capturing the tag (e.g. following separation of unbound probe from the sample) and correlating presence of the tag with presence of probe bound to amplification product, and hence the presence of S. enterica subsp. IIIa and/or IIIb.

In one embodiment, the tag is captured using a capture molecule, which may be attached (e.g. coated) onto a substrate or solid support, such as a membrane or magnetic bead.

Capture methods employing magnetic beads are advantageous because the beads (plus captured, tagged probe bound to amplification product) can easily be separated from the sample, using conventional techniques known in the art.

Examples of suitable tags include biotin and streptavidin. In this regard, a biotin tag may be captured using streptavidin, which may be coated onto a substrate or support such as a bead (for example a magnetic bead) or membrane. Likewise, a streptavidin tag may be captured using biotin, which may be coated onto a substrate or support such as a bead (for example a magnetic bead) or membrane. Other exemplary pairs of tags and capture molecules include receptor/ligand pairs and antibody/antigen (or hapten or epitope) pairs.

Thus, in one embodiment, the probe is tagged with biotin, and the detection step comprises contacting the sample with a streptavidin-coated magnetic bead, which captures the biotin-tagged probe bound to amplification product. The magnetic bead (plus captured, tagged probe bound to amplification product) is then separated from the sample, thereby separating the amplification product from the sample. The amplification product can then be detected by any known means.

In one embodiment, the probe comprises a minor groove binder component.

In one embodiment, the probe comprises reporter and quencher fluorophores. In one embodiment, a reporter fluorophore is located at or near one end of the probe and a quencher fluorophore is located at or near the opposite end of the probe. For example, the reporter fluorophore may be located at or near the 5′ end of the probe and the quencher fluorophore may be located at or near the 3′ end of the probe (or vice versa).

Suitable reporter fluorophores include FAM, VIC/JOE/Yakima Yellow, NED/TAMRA/Cy3, ROX/TR and Cy5.

Suitable quencher fluorophores include TAMRA and the Black Hole quencher series.

In one embodiment, cleavage of the probe separates the reporter and quencher fluorophores. In one embodiment, separation of the reporter and quencher fluorophores results in a detectable fluorescent signal, or results in a detectable change in a fluorescent signal. Thus, in one embodiment, the detection step comprises (e.g. after separating un-hybridised probe from the sample) cleaving the hybridised probe to separate the reporter and quencher fluorophores; and detecting a fluorescent signal or detecting a change in a fluorescent signal; wherein said fluorescent signal, or change in fluorescent signal, is indicative of the presence of the amplification product, and hence the presence of S. enterica subsp. IIIa and/or IIIb.

By way of example, bound probe may be cleaved by an extending polymerase with 5′ to 3′ exonuclease activity, as may occur in a real-time PCR assay, such as a Taqman® assay.

In one embodiment of the present invention, the Taqman® system for amplifying and detecting a target nucleic acid sequence is employed. For optimal performance of the Taqman® assay, the length of the amplification product is less than 200 nucleotides, such as less than 150 nucleotides, and the probe may have a melting temperature higher (e.g. about 10° C. higher) than the primers. Typically this results in the probe being several nucleotides longer than the primers.

In one aspect, the probe is immobilised onto a support or platform. Immobilising the probe provides a physical location for the probe, and may serve to fix the probe at a desired location and/or facilitate recovery or separation of probe. The support may be a rigid solid support made from, for example, glass or plastic, such as a bead (for example a magnetic bead). Alternatively, the support may be a membrane, such as nylon or nitrocellulose membrane. 3D matrices are also suitable supports for use with the present invention—e.g. polyacrylamide or PEG gels.

Immobilisation to a support/platform may be achieved by a variety of conventional means. By way of example, immobilisation onto a support such as a nylon membrane may be achieved by UV cross-linking. Biotin-labelled molecules (e.g. probes) may be bound to streptavidin-coated substrates (and vice-versa), and molecules prepared with amino linkers may be immobilised onto silanised surfaces. Another means of immobilising a probe is via a poly-T tail or a poly-C tail, for example at the 3′ or 5′ end.

In one aspect, the amplification product is a double-stranded nucleic acid molecule and is detected by a method comprising melt curve analysis. Melting curve analysis is an assessment of the dissociation characteristics of double-stranded nucleic acid (e.g. DNA) during heating.

In one aspect, the amplification product is detected by a method comprising contacting the sample with an enzyme (such as a restriction endonuclease) that digests the amplification product, and identification of digestion products.

In this aspect, the restriction endonuclease recognises a restriction site that is located within the sequence of the amplification product.

In this embodiment, the presence of digestion products confirms that amplification product is present and hence confirms the presence of S. enterica subsp. IIIa and/or IIIb. In contrast, the absence of digestion products confirms that amplification product is absent, and hence confirms the absence of S. enterica subsp. IIIa and/or IIIb.

The digestion products may be detected by any known means, for example by a method comprising any of the detection techniques discussed above. In one embodiment, the digestion products of the amplification product are detected by virtue of their size, for example by a method comprising gel electrophoresis.

In one embodiment, the method comprises contacting the sample with a second pair of forward and reverse oligonucleotide primers, wherein said forward and reverse primers act as an internal amplification control to confirm presence of Salmonella sp. In one embodiment, the control primers hybridise to target nucleic acid sequences located within a nucleic acid sequence that is specific to all Salmonella sp., such as the Salmonella ttrRSBCA locus. In one embodiment, said method comprises extending said forward and reverse control primers to generate a control amplification product, and detecting the control amplification product. In one embodiment, detection of the control amplification product comprises contacting the sample with a control oligonucleotide probe that forms a hybridisation complex with the control amplification product, if present, and detecting the hybridisation complex.

In one embodiment, the sample is contacted with the control primers and/or control probe simultaneously with (in parallel with or in combination with) the forward and reverse primers and/or probe of the invention, or sequentially with (prior to or after) the forward and reverse primers and/or probe of the invention.

The method of the present invention enables quantitative estimates of S. enterica subsp. IIIa and/or IIIb bacterial load to be determined. Determining bacterial load has many useful applications, such as for clinical guidance and for determining therapy, for patient management and for assessing vaccine efficacy.

In one aspect, measuring the amount of amplification product detected enables quantification of the amount of S. enterica subsp. IIIa and/or IIIb nucleic acid in a sample.

In one embodiment, the amplification product is labelled and the amount of amplification product is measured by detecting the label and measuring the amount of label. In one embodiment, the amplification product is tagged and the amount of amplification product is quantified by capturing the tag and measuring the amount of captured tag.

In one embodiment, the amplification product is hybridised with an oligonucleotide probe, and the amount of amplification product is measured by measuring the amount of probe-amplification product hybridisation complexes. In one embodiment, the probe is tagged or labelled, and the amount of probe-amplification product hybridisation complexes is measured by detecting the label or capturing the tag (e.g. after separating un-hybridised probe from the sample), and measuring the presence (and optionally the quantity) of label or captured tag, wherein the presence of the label or tag is indicative of the presence of the hybridisation complex.

In one embodiment, the amount of probe-amplification product hybridisation complexes is measured by detecting (and optionally quantifying) a fluorescent signal or a change in a fluorescent signal, wherein said fluorescent signal or a change in a fluorescent signal is indicative of the presence of the hybridisation complex. In one embodiment, said fluorescent signal (or change therein) is generated by separation of reporter and quencher fluorophores, for example by cleavage of a probe to which said fluorophores are attached.

In one embodiment, the amplification product is digested with a restriction endonuclease, and the amount of amplification product is measured by detecting digestion products of the amplification product, and measuring the amount of digestion product.

In one aspect, the present invention provides an in vitro method for quantitating the bacterial load of S. enterica subsp. IIIa and/or IIIb in a sample of interest, comprising: (a) carrying out a detection method according to the present invention on said sample of interest; and (b) carrying out said method on a test sample having a predetermined bacterial load of S. enterica subsp. IIIa and/or IIIb; and (c) comparing the amount of amplification product detected from the sample of interest with the amount of amplification product detected from the test sample; and thereby quantitating the bacterial load of S. enterica subsp. IIIa and/or IIIb in the sample of interest.

In another aspect, the method of the present invention is useful for determining efficacy of a course of treatment for S. enterica subsp. IIIa and/or IIIb infection over a period of time, for example a course of therapy, such as drug or vaccine therapy.

Thus, in one aspect, the present invention provides an in vitro method of determining the efficacy of an anti-S. enterica subsp. IIIa and/or IIIb therapy (such as an anti-S. enterica subsp. IIIa and/or IIIb drug) over the course of a period of therapy, comprising: (a) carrying out a detection method according to the present invention on a first sample obtained at a first time point within or prior to the period of therapy; (b) carrying out said method on one or more samples obtained at one or more later time points within or after the period of therapy; and (c) comparing the amount of amplification product detected from the first sample with the amount of amplification product detected from the one or more later samples; and thereby determining drug efficacy over the course of the period of drug therapy.

In one embodiment, a reduction in the quantity of amplification product detected from the one or more later samples, as compared with the quantity of amplification product detected from the first sample, indicates efficacy of the drug against S. enterica subsp. IIIa and/or IIIb.

In another aspect, the present invention is useful for determining the efficacy of a vaccine against infection with S. enterica subsp. IIIa and/or IIIb.

Thus, in one aspect, the present invention provides an in vitro method of determining the efficacy of a vaccine against S. enterica subsp. IIIa and/or IIIb, comprising: (a) carrying out a detection method according to the present invention on a first sample obtained from a patient at a first time point prior to vaccination; (b) carrying out said method on a sample obtained from said patient at one or more later time points after vaccination and following challenge with S. enterica subsp. IIIa and/or IIIb; and (c) comparing the amount of amplification product detected from the first sample with the amount of amplification product detected from the one or more later samples; and thereby determining vaccine efficacy.

In one embodiment, a reduction in the quantity of amplification product detected from the one or more later samples, as compared with the quantity of amplification product detected from the first sample, indicates efficacy of the vaccine against infection with S. enterica subsp. IIIa and/or IIIb.

The invention also provides reagents such as forward primers, reverse primers, probes, combinations thereof, and kits comprising said reagents, for use in the above-described methods of the present invention.

In one embodiment, the sequence of the forward and/or reverse oligonucleotide primer and/or probe does not comprise or consist of the entire nucleic acid sequence of SEQ ID NO: 1, 11 or 12, or the complement thereof.

In one aspect, the invention provides a forward oligonucleotide primer as defined above, which hybridises to a target nucleic acid sequence located within the complement of SEQ ID NO: 1, 11 or 12.

In one embodiment, the forward primer hybridises to a target sequence located between residues 1-2050 of the complement of SEQ ID NO: 1, 11 or 12. Within this range, the forward primer target sequence may be located from residue 200, 400, 600, 800, 1000, 1100, 1200, 1300, 1400, 1500, 1600, 1700, 1800, 1900, 1910, 1920, 1930, 1940, 1945, 1950, 1951, 1952, 1953, 1954, 1955, 1956, 1957 or 1958 of the complement of SEQ ID NO: 1, 11 or 12. Within this range, the forward primer target sequence may be located up to residue 2040, 2030, 2020, 2010, 2000, 1990, 1985, 1984, 1983, 1982, 1981, 1980, 1979, 1978 or 1977 of the complement of SEQ ID NO: 1, 11 or 12. For example, the forward primer may hybridise to a target sequence located between residues 1930-2000, such as between residues 1950-1980 of the complement of SEQ ID NO: 1, 11 or 12. In one embodiment, the forward primer target sequence is defined by residues 1958-1977 of the complement of SEQ ID NO: 1, 11 or 12.

In one embodiment, said forward primer target nucleic acid sequence comprises (or consists of) a nucleotide sequence that is at least 75% identical to (such as 80, 85, 86, 87, 88, 89, 90, 91, 92, 93, 94, 95, 96, 97, 98, 99 or 100% identical to) SEQ ID NO: 2, or a fragment thereof as defined above. In one embodiment, said forward primer target nucleic acid sequence is specific to S. enterica subsp. IIIa and/or IIIb.

In one embodiment, the forward primer comprises (or consists of) a nucleotide sequence having at least 75% identity to (such as 80, 85, 86, 87, 88, 89, 90, 91, 92, 93, 94, 95, 96, 97, 98, 99 or 100% identity to) a nucleotide sequence of SEQ ID NO: 4.

In one embodiment, the forward primer comprises (or consists of) a fragment of SEQ ID NO: 4 (or a sequence variant thereof as defined above) wherein said fragment comprises at least 15 consecutive nucleotides thereof. In one embodiment, said fragment comprises at least 16, 17, 18 or 19 consecutive nucleotides thereof.

In one aspect, the invention provides a reverse oligonucleotide primer as defined above, which hybridises to a target nucleic acid sequence located within SEQ ID NO: 1, 11 or 12.

In one embodiment, the reverse primer hybridises to a target sequence located between residues 2050-2520 of SEQ ID NO: 1, 11 or 12, for example in a region located from residue 2055, 2060, 2061, 2062, 2063, 2064, 2065, 2066, 2067 or 2068 of SEQ ID NO: 1, 11 or 12, for example in a region up to residue 2500, 2400, 2300, 2200, 2150, 2140, 2130, 2120, 2110, 2100, 2095, 2094, 2093, 2092, 2091, 2090, 2089, 2088 or 2087 of SEQ ID NO: 1, 11 or 12. For example, the reverse primer may hybridise to a target sequence located between residues 2050-2110, such as residues 2060-2095 of SEQ ID NO: 1, 11 or 12. In one embodiment, the reverse primer target sequence is defined by residues 2068-2087 of SEQ ID NO: 1, 11 or 12.

In one embodiment, said reverse primer target nucleic acid sequence comprises (or consists of) a nucleotide sequence that is at least 75% identical to (such as 80, 85, 86, 87, 88, 89, 90, 91, 92, 93, 94, 95, 96, 97, 98, 99 or 100% identical to) a nucleotide sequence of SEQ ID NO: 3. In one embodiment, said target nucleic acid sequence is specific to S. enterica subsp. IIIa and/or IIIb.

In one embodiment, the reverse primer comprises (or consists of) a nucleotide sequence having at least 75% identity to (such as 80, 85, 86, 87, 88, 89, 90, 91, 92, 93, 94, 95, 96, 97, 98, 99 or 100% identity to) a nucleotide sequence of SEQ ID NO: 5.

In one embodiment, the reverse primer comprises (or consists of) a fragment of SEQ ID NO: 5 (or a sequence variant thereof as defined above) wherein said fragment comprises at least 15 consecutive nucleotides thereof. In one embodiment, said fragment comprises at least 16, 17, 18 or 19 consecutive nucleotides thereof.

In one embodiment, the forward primer and/or the reverse primer comprise a tag or label, as described above.

The present invention further provides a pair of forward and reverse oligonucleotide primers, comprising a forward primer as defined above and a reverse primer as defined above.

The present invention also provides a probe, such as an oligonucleotide probe as defined above.

In one embodiment, the probe hybridises to a target binding sequence located within the complement of SEQ ID NO: 1, 11 or 12. In one embodiment, the probe binds a target nucleotide sequence located between residues 1900-2100 of the complement of SEQ ID NO: 1, 11 or 12. Within this range, the probe target sequence may be located in a region from residue 1910, 1920, 1930, 1940, 1950, 1960, 1965, 1970, 1975, 1976, 1977, 1978, 1979, 1980 or 1981 of the complement of SEQ ID NO: 1, 11 or 12. Within this range, the probe target sequence may be located in a region up to residue 2090, 2080, 2070, 2060, 2050, 2040, 2030, 2020, 2015, 2010, 2009, 2008, 2007, 2006, 2005 or 2004 of the complement of SEQ ID NO: 1, 11 or 12. For example, the forward primer may hybridise to a target sequence located between residues 1960-2020, such as residues 1975-2010 of the complement of SEQ ID NO: 1, 11 or 12. In one embodiment, the forward primer target sequence is defined by residues 1981-2004 of the complement of SEQ ID NO: 1, 11 or 12.

In one embodiment, the target binding sequence for the probe comprises (or consists of) a target sequence that is at least 75% identical (such as at least 80, 85, 90, 91, 92, 93, 94, 95, 96, 97, 98, 99 or 100% identical) to a nucleotide sequence of SEQ ID NO: 6, or to a fragment thereof having at least 19 consecutive nucleotides thereof (such as at least 20, 21, 22 or 23 consecutive nucleotides thereof).

In one embodiment, said probe comprises (or consists of) a nucleotide sequence having at least 75% identity to (such as at least 80, 85, 86, 87, 88, 89, 90, 91, 92, 93, 94, 95, 96, 97, 98, 99 or 100% identity to) a nucleotide sequence of SEQ ID NO: 7, or a fragment thereof having at least 18 (such as at least 19, 20, 21, 22 or 23) consecutive nucleotides thereof.

In one embodiment, the probe comprises a tag or label, as described above, or reporter and quencher fluorophores, as described above.

The present invention also provides a kit for detecting S. enterica subsp. IIIa and/or IIIb bacteria in a sample, comprising a pair of forward and reverse oligonucleotide primers as defined above.

The kit optionally comprises amplification reagents such as a polymerase (e.g. a polymerase having 5′-3′ exonuclease activity such as Tag polymerase) and/or DNA precursors.

The kit optionally comprises reagents for detection of the amplification product. In one embodiment, reagents for detection of the amplification product comprise an oligonucleotide probe as described above, which hybridises to said amplification product. In one embodiment, reagents for detection of the amplification product comprise an enzyme such as a restriction endonuclease (such as HhaI) that digests the amplification product, as described above.

In one embodiment, the invention further provides a method for detecting a Salmonella enterica subsp. I (enterica) in a sample. In one embodiment, said method is based on detection of a nucleic acid sequence (such as a gene sequence) that is specific to that Salmonella enterica subsp. I (enterica).

Existing assays for detecting S. enterica subsp. I (e.g. based on detecting the centrisome 7 genomic island or shdA) are problematic, because they are not fully S. enterica subsp. I inclusive (i.e. they are often less than 90% sensitive and do not detect a significant proportion of S. enterica subsp. I strains).

Other known assays for S. enterica subsp. I are problematic because they detect other Salmonella subsp. (i.e. these known assays are not specific for S. enterica subsp. I).

In one embodiment, the invention provides a method for detecting Salmonella subsp. I in a sample, based on detection of the hilA gene. In one embodiment, the method is based on detection of one or more regions of the hilA gene that are specific to S. enterica subsp. I and are conserved between strains of S. enterica subsp. I.

In this regard, we have collected Salmonella hilA gene sequences available in public databases and have ourselves sequenced a number of hilA genes from representatives of all the Salmonella subspecies. Using this data, we have identified regions of the hilA gene that are specific to S. enterica subsp. I and are conserved between strains of S. enterica subsp. I. To the best of our knowledge, these specific regions of the hilA gene have not previously been targeted.

In one embodiment, the invention provides a method for detecting S. enterica subsp. I (enterica) in a sample, the method comprising:
(a) contacting the sample with a pair of forward and reverse oligonucleotide primers, wherein said forward and reverse primers hybridise to target nucleic acid sequences located within the hilA gene of S. enterica subsp. I, or the complement thereof;
(b) extending said forward and reverse primers to generate an amplification product; and
(c) detecting the amplification product.

In one embodiment, discussed below, the amplification product is detected by a method comprising contacting the sample with an oligonucleotide probe that forms a hybridisation complex with the amplification product, if present; and detecting the hybridisation complex.

This method advantageously provides a highly sensitive, specific, rapid and robust molecular diagnostic assay for Salmonella enterica subsp. I (enterica), which has the potential to replace the existing laborious and long turnaround biochemical assays presently being used.

In one embodiment, the Salmonella enterica subsp. I assay shows 100% specificity and 99.7% sensitivity. In one embodiment, the assay does not detect any Hafnia, Citrobacter, Enterobacter, Escherichia or Proteus spp. In one embodiment, the assay identifies S. enterica subsp. I in less than 2 hours, compared to an average minimum turnaround time of 14-28 days for full routine biochemistry and serology-based identification methods.

In one embodiment, the assay is useful for screening clinical, veterinary, food or research-based samples for S. enterica subsp. I, to aid monitoring, or to monitor epidemiology and natural history of the disease, along with other potential research.

Several versions of the sequence for the hilA gene of S. enterica subsp. I are publically available, two of which are represented herein by SEQ ID NOs: 13 and 14. SEQ ID NO: 13 corresponds to the hilA gene of S. enterica subsp. enterica serovar Typhimurium LT2 publically available under GenBank Accession number. AE008831 (nucleotides 8999-10660). SEQ ID NO: 14 corresponds to the hilA gene of S. enterica subsp. enterica serovar Typhimurium publically available under GenBank Accession number U25352 (nucleotides 847-2508).

Other complete subspecies I hilA sequences in GenBank include:

AM933172=S. enterica subsp. enterica serovar Enteritidis str. P125109 complete genome (hilA coding sequence=2904516-2906177 nt); CP001120=S. enterica subsp. enterica serovar Heidelberg str. SL476, complete genome (hilA coding sequence=2994370-2996031 nt); CP000886=S. enterica subsp. enterica serovar Paratyphi B str. SPB7, complete genome (hilA coding sequence=2982838-2984499 nt); AM933173=S. enterica subsp. enterica serovar Gallinarum str. 287/91 complete genome (hilA coding sequence=2895320-2896981 nt); AE017220=S. enterica subsp. enterica serovar Choleraesuis str. SC-B67, complete genome (hilA coding sequence=2973384-2975045 nt); P001144=S. enterica subsp. enterica serovar Dublin str. CT_{—}02021853, complete genome (hilA coding sequence=3064267-3065914 nt); CP001113=S. enterica subsp. enterica serovar Newport str. SL254, complete genome (hilA coding sequence=2996360-2998021 nt); AL627276=S. enterica serovar Typhi (Salmonella typhi) strain CT18, complete chromosome; segment 12/20 (hilA coding sequence=169866-171527 nt); AE014613=S. enterica subsp. enterica serovar Typhi Ty2, complete genome (hilA coding sequence=2857723-2859384 nt); X80892=S. enterica subsp. enterica serovar typhi genes iagA and iagB (hilA coding sequence=98-1759 nt); FM200053=S. enterica subsp. enterica serovar Paratyphi A str. AKU_{—}12601 complete genome (hilA coding sequence=2829938-2831599 nt); CP000026=S. enterica subsp. enterica serovar Paratyphi A str. ATCC 9150 (hilA coding sequence=2834402-2836063 nt); CP001127=S. enterica subsp. enterica serovar Schwarzengrund str. CVM19633, complete genome (hilA coding sequence=2927061-2928722 nt); and CP001138=S. enterica subsp. enterica serovar Agona str. SL483, complete genome (hilA coding sequence=2928382-2930043 nt).

The publically available hilA sequences share approximately 97% identity over their entire length. In this regard, SEQ ID NOs: 13 and 14 share approximately 99% identity over their entire length.

Thus, in one embodiment, the hilA gene of S. enterica subsp. I comprises (or consists of) a nucleotide sequence having at least 90% identity (such as at least 91, 92, 93, 94, 95, 96, 97, 98, 99 or 100% identity) to a nucleotide sequence selected from SEQ ID NOs: 13 or 14 or a hilA nucleotide sequence deposited under the Accession numbers recited above.

In one embodiment, the method comprises contacting the sample with a pair of forward and reverse oligonucleotide primers, wherein said forward and reverse primers hybridise to target nucleic acid sequences located within a nucleotide sequence having at least 90% identity (such as at least 91, 92, 93, 94, 95, 96, 97, 98, 99 or 100% identity) to a hilA nucleotide sequence selected from SEQ ID NOs: 13 or 14 or a hilA nucleotide sequence deposited under the Accession numbers recited above.

All S. enterica subspecies I have the hilA gene. The hilA gene encodes a transcriptional activator that regulates expression of Salmonella virulence genes in response to environmental stimuli.

The Applicant has unexpectedly identified a region of the hilA gene that is specific to S. enterica subsp. I, and conserved between strains of S. enterica subsp. I.

In one embodiment, the method comprises amplification and detection of a region of the hilA gene that is specific to strains of S. enterica subsp. I. In one embodiment, the method comprises amplification and detection of a region of the hilA gene that is distinct from the hilA gene of all other Enterobacteriaceae.

Throughout this application, the term “S. enterica subsp. I” is equivalent to the term “S. enterica subsp. enterica”.

In one embodiment, the sample is (or is derived from) a clinical, veterinary, food, water, environmental or faecal sample or bacterial culture or DNA extract.

In one embodiment, the target nucleic acid sequence to which the forward primer hybridises is specific to S. enterica subsp. I. In one embodiment, the target nucleic acid sequence to which the reverse primer hybridises is specific to S. enterica subsp. I.

In one embodiment, extension of the forward and reverse primers generates an amplification product comprising a nucleic acid sequence that is specific to S. enterica subsp. I.

In general, a reverse primer is designed to hybridise to a target nucleic acid sequence within the coding (sense) strand of a target nucleic acid, and a forward primer is designed to hybridise to a target nucleic acid sequence within the complementary (i.e. anti-sense) strand of the target nucleic acid.

The term “complement of a nucleic acid sequence” refers to a nucleic acid sequence having a complementary nucleotide sequence as compared to a reference nucleotide sequence.

In one embodiment, the forward primer hybridises to a target nucleic acid sequence (a ‘forward primer target sequence’) located within the complement of SEQ ID NO: 13 or 14.

In one embodiment, the forward primer target sequence has a length in the range of 10-40 consecutive nucleotides of the complement of SEQ ID NO: 13 or 14, such as at least 11, 12, 13, 14, 15, 16, 17, 18, 19, 20, 21, 22, 23 or 24 consecutive nucleotides of the complement of SEQ ID NO: 13 or 14; such as up to 39, 38, 37, 36, 35, 34, 33, 32, 31, 30, 29, 28, 27, 26, 25 or 24 consecutive nucleotides of the complement of SEQ ID NOs: 13 or 14. For example, the reverse primer target sequence may have a length of 20-30 consecutive nucleotides of the complement of SEQ ID NO: 13 or 14, such as a length of 22-26 consecutive nucleotides of the complement of SEQ ID NO: 13 or 14, for example a length of about 24 consecutive nucleotides of the complement of SEQ ID NO: 13 or 14.

In one embodiment, the forward primer target sequence is specific to S. enterica subsp. I.

In one embodiment, the forward primer hybridises to a target sequence located between residues 1-1560 of the complement of SEQ ID NO: 13 or 14. Within this range, the forward primer target sequence may be located in a region from residue 200, 400, 600, 800, 1000, 1200, 1300, 1350, 1400, 1450, 1460, 1470, 1480, 1490, 1500, 1505, 1506, 1507, 1508, 1509, 1510 or 1511 of the complement of SEQ ID NO: 13 or 14. Within this range, the forward primer target sequence may be located in a region up to residue 1555, 1550, 1545, 1540, 1539, 1538, 1537, 1536, 1535 or 1534 of the complement of SEQ ID NO: 13 or 14. For example, the forward primer may hybridise to a target sequence located between residues 1475-1555 of the complement of SEQ ID NO: 13 or 14, such as a target sequence located between residues 1500-1550 of the complement of SEQ ID NO: 13 or 14. In one embodiment, the forward primer target sequence is defined by residues 1511-1534 of the complement of SEQ ID NO: 13 or 14.

For the avoidance of doubt, the above numbering system applied to the nucleic acid residues of the complementary strand of SEQ ID NOs: 13 or 14 is based on the numbering of the nucleic acids of SEQ ID NOs: 13 or 14 to which they are complementary.

In one embodiment, the forward primer hybridises to a target nucleic acid sequence that comprises (or consists of) SEQ ID NO: 15 (shown below) or a nucleotide sequence that is at least 75% identical thereto (such as 80, 85, 90, 91, 92, 93, 94, 95, 96, 97, 98, 99 or 100% identical thereto), or a fragment thereof.

| | Target nucleic acid sequence |
|---|---|
| SEQ ID NO: | for forward primer (5′ to 3′) |
| 15 | TCG CAT ATC TAT TAT TAG GCC CTA |
| 16 | TCG CAT AAC TAT TAT TAG GCC CTA |

In one embodiment, a fragment of SEQ ID NOs: 15 or 16 (or sequence variants thereof as defined above), comprises (or consists of) at least 15 consecutive nucleotides thereof, for example at least 16, 18, 20, 21, 22 or 23 consecutive nucleotides thereof.

In one embodiment, the reverse primer hybridises to a target nucleic acid sequence (a ‘reverse primer target sequence’) located within SEQ ID NO: 13 or 14.

In one embodiment, the reverse primer target sequence has a length in the range of 10-40 consecutive nucleotides of SEQ ID NO: 13 or 14, such as at least 11, 12, 13, 14, 15, 16, 17, 18, 19, 20, 21, 22 or 23 consecutive nucleotides of SEQ ID NO: 13 or 14, such as up to 38, 35, 33, 32, 31, 30, 29, 28, 27, 26, 25, 24 or 23 consecutive nucleotides of SEQ ID NO: 13 or 14. For example, the reverse primer target sequence may have a length of 18-30 consecutive nucleotides of SEQ ID NO: 13 or 14, such as a length of 20-25 consecutive nucleotides of SEQ ID NO: 13 or 14, for example a length of about 23 consecutive nucleotides of SEQ ID NO: 13 or 14.

In one embodiment, the reverse primer target sequence is specific to S. enterica subsp. I.

In one embodiment, the reverse primer hybridises to a target sequence located between residues 1560-1662 of SEQ ID NO: 13 or 14. Within this range, the forward primer target sequence may be located in a region from residue 1561, 1562, 1563, 1564, 1565, 1566, 1567 or 1568 of SEQ ID NO: 13 or 14. Within this range, the forward primer target sequence may be located in a region up to residue 1660, 1650, 1640, 1630, 1625, 1620, 1615, 1610, 1605, 1600, 1595, 1594, 1593, 1592, 1591 or 1590 of SEQ ID NO: 13 or 14. For example, the reverse primer may hybridise to a target sequence located between residues 1560-1625 of SEQ ID NO: 13 or 14, such as a target sequence located between residues 1565-1600 of SEQ ID NO: 13 or 14. In one embodiment, the reverse primer target sequence is defined by residues 1568-1590 of SEQ ID NO: 13 or 14.

In one embodiment, the reverse primer hybridises to a target nucleic acid sequence that comprises (or consists of) a nucleotide sequence that is at least 75% identical to (such as 80, 85, 90, 91, 92, 93, 94, 95, 96, 97, 98, 99 or 100% identical to) a nucleotide sequence of SEQ ID NO: 17 or 18 (shown below), or a fragment thereof.

| | Target nucleic acid sequence |
|---|---|
| SEQ ID NO: | for reverse primer (5′ to 3′) |
| 17 | TAA GGT GTA AAA GAG CCG TTA TC |
| 18 | CAA GGT GTA AAA GAG CCG TTA TC |

In one embodiment, a fragment of SEQ ID NO: 17 or 18 (or sequence variants thereof as defined above), comprises (or consists of) at least 15, 16, 17, 18, 19, 20, 21 or 22 consecutive nucleotides thereof.

In one embodiment, the forward primer is 15-30 nucleotides long, such as at least 16, 17, 18, 19, 20, 21, 22, 23 or 24 nucleotides long, such as up to 29, 28, 27, 26, 25 or 24 nucleotides long. For example, the reverse primer may be 22-26 nucleotides long, such as about 24 nucleotides long.

In one embodiment, the reverse primer is 15-30 nucleotides long, such as at least 16, 17, 18, 19, 20, 21, 22 or 23 nucleotides long, such as up to 29, 28, 27, 26, 25, 24 or 23 nucleotides long. For example, the reverse primer may be 20-25 nucleotides long, such as about 23 nucleotides long.

In one embodiment, the forward primer comprises (or consists of) a nucleotide sequence having at least 75% identity to (such as at least 80, 85, 90, 91, 92, 93, 94, 95, 96, 97, 98, 99 or 100% identity to) a nucleotide sequence of SEQ ID NO: 19 or 20 (shown below). Conservative substitutions may be useful in this regard.

| SEQ ID NO: | Forward primer sequence (5′ to 3′) |
|---|---|
| 19 | AGC GTA TAG ATA ATA ATC CGG GAT |
| 20 | AGC GTA TTG ATA ATA ATC CGG GAT |

Variants of SEQ ID NO: 19 or 20 may alternatively be defined by reciting the number of nucleotides that differ between the variant sequences and SEQ ID NO: 19 or 20. Thus, in one embodiment, the forward primer may comprise (or consist of) a nucleotide sequence that differs from SEQ ID NO: 19 or 20 at no more than 5 nucleotide positions, for example at no more than 4, 3, 2 or 1 nucleotide positions. Conservative substitutions may be useful in this regard.

Fragments of the above-mentioned forward primer sequence (and sequence variants thereof as defined above) may also be employed. In one embodiment, the forward primer may comprise (or consist of) a fragment of SEQ ID NO: 19 or 20 (and sequence variants thereof as defined above), wherein said fragment may comprise at least 15 consecutive nucleotides thereof, such as at least 16, 17, 18, 19, 20, 21, 22 or 23 consecutive nucleotides thereof.

In one embodiment, the reverse primer comprises (or consists of) a nucleotide sequence having at least 75% identity to (such as at least 80, 85, 90, 91, 92, 93, 94, 95, 96, 97, 98, 99 or 100% identity to) a nucleotide sequence of SEQ ID NO: 21 or 22. Conservative substitutions may be useful in this regard.

| SEQ ID NO: | Reverse primer sequence (5′ to 3′) |
|---|---|
| 21 | ATT CCA CAT TTT CTC GGC AAT AG |
| 22 | GTT CCA CAT TTT CTC GGC AAT AG |

Variants of SEQ ID NO: 21 or 22 may alternatively be defined by reciting the number of nucleotides that differ between the variant sequences and SEQ ID NO: 21 or 22. In one embodiment, the reverse primer may comprise (or consist of) a nucleotide sequence that differs from SEQ ID NO: 21 or 22 at no more than 5 nucleotide positions, for example at no more than 4, 3, 2 or 1 nucleotide positions. In this regard, conservative substitutions may be useful.

Fragments of the above-mentioned reverse primer sequences (and sequence variants thereof as defined above) may also be employed. In one embodiment, the reverse primer may comprise (or consist of) a fragment of SEQ ID NO: 21 or 22 (and sequence variants thereof as defined above), wherein said fragment may comprise at least 15 consecutive nucleotides thereof, such as at least 16, 17, 18, 19, 20, 21 or 22 consecutive nucleotides thereof.

In one embodiment, the forward primer is sequence-specific and hybridises specifically to the forward primer target nucleic acid sequence within SEQ ID NO: 13 or 14. In one embodiment, the reverse primer is sequence-specific and hybridises specifically to the reverse primer target nucleic acid sequence within SEQ ID NO: 13 or 14. In one embodiment, the binding conditions are such that a high level of specificity is provided. In one embodiment, the melting temperature (Tm) of the forward and reverse primers is in excess of 64° C., for example about 66° C.

In one embodiment, the forward primer and/or the reverse primer comprises a tag or label. In one embodiment, said tag or label is incorporated into the amplification product when the primer is extended. The tag or label may be located at the 5′ or 3′ end of the forward and/or reverse primer, for example at the 5′ end of the reverse primer.

Examples of suitable labels and tags are as described above with respect to the primers for the lacZ assay (S. enterica subsp. IIIa and/or IIIb). Likewise, the conventional amplification techniques and platforms described above with respect to detection of the lacZ nucleic acid sequence (S. enterica subsp. IIIa and/or IIIb) are also suitable for amplifying target hilA nucleic acid sequence (S. enterica subsp. I).

In one embodiment, the hilA amplification product is in the range of 30-150 nucleotides, such as at least 40, 50, 60, 65, 70, 75 or 80 nucleotides, such as up to 140, 130, 120, 110, 100, 95, 90, 85 or 80 nucleotides. For example, the amplification product may be in the range of 40-120 nucleotides, such as in the range of 60-100 nucleotides, such as about 80 nucleotides.

The detection step may be carried out by any known means. The detection techniques described above with respect to detection of the lacZ amplification product (S. enterica subsp. IIIa/IIIb) are also suitable for detecting the hilA amplification product (S. enterica subsp. I). By way of example, as discussed above, the detection step may comprise detecting a tag or label (via any of the techniques defined above, e.g. capture methods employing magnetic beads).

In one embodiment, the nucleic acid sequence of the amplification product is determined, by any known means (e.g. as described above).

In one aspect, the hilA amplification product is detected by a method comprising contacting the sample with an oligonucleotide probe under conditions allowing the formation of hybridisation complexes between the probe and the amplification product, and detecting the hybridisation complexes.

In one embodiment, the probe is specific for the amplification product.

In one embodiment, the probe is 15-40 nucleotides long, such as at least 16, 17, 18, 19, 20, 21, 22, 23 or 24 nucleotides long, such as up to 39, 38, 37, 36, 35, 34, 33, 32, 31, 30, 29, 28, 27, 26, 25 or 24 nucleotides long. For example, the probe may be 20-30 nucleotides long, such as 22-26 nucleotides long. In one embodiment, the probe is about 24 nucleotides long.

In one embodiment, the target nucleotide sequence to which the probe hybridises within the amplification product is 15-40 nucleotides long, such as at least 16, 17, 18, 19, 20, 21, 22, 23 or 24 nucleotides long, such as up to 39, 38, 37, 36, 35, 34, 33, 32, 31, 30, 29, 28, 27, 26, 25 or 24 nucleotides long. For example, the target nucleotide sequence for the probe may be 20-30 nucleotides long, such as 22-26 nucleotides long. In one embodiment, the probe binds a target nucleotide sequence that is about 24 nucleotides long.

Probes for detecting the hilA amplification product are designed and screened by conventional methods—e.g. as described above with respect to probes for detecting the lacZ amplification product (S. enterica subsp. IIIa/IIIb). In one embodiment, hybridisation of the probe to the hilA amplification product occurs under “stringent conditions” (as defined above). In one embodiment, the Tm of the hilA probes, at a salt concentration of about 0.02M or less at pH 7, is above 65° C., such as about 74° C.

In one embodiment, the target binding sequence for the probe is located within the complement of SEQ ID NO: 13 or 14. In one embodiment, the probe binds a target nucleotide sequence located between residues 1521-1580 of the complement of SEQ ID NO: 13 or 14. Within this range, the probe target sequence may be located in a region from residue 1525, 1530, 1531, 1532, 1533, 1534, 1535, 1536 or 1537 of the complement of SEQ ID NO: 13 or 14. Within this range, the probe target sequence may be located in a region up to residue 1575, 1570, 1569, 1568, 1567, 1566, 1565, 1564, 1563, 1562, 1561 or 1560 of the complement of SEQ ID NO: 13 or 14. For example, the forward primer may hybridise to a target sequence located between residues 1530-1570 of the complement of SEQ ID NO: 13 or 14, such as a target sequence located between residues 1535-1565 of the complement of SEQ ID NO: 13 or 14. In one embodiment, the forward primer target sequence is defined by residues 1537-1560 of the complement of SEQ ID NO: 13 or 14.

In one embodiment, the target binding sequence for the probe comprises (or consists of) a nucleotide sequence that is at least 75% identical (such as at least 80, 85, 90, 91, 92, 93, 94, 95, 96, 97, 98, 99 or 100% identical) to a nucleotide sequence of SEQ ID NO: 23 (shown below), or to a fragment thereof having at least 18 nucleotides (such as at least 19, 20, 21, 22 or 23 nucleotides).

| SEQ ID NO: | Probe target sequence (5′ to 3′) |
|---|---|
| 23 | GTG GGC AAC CAG CAC TAA CGG TAA |

In one aspect, the oligonucleotide probe comprises (and may consist of) a nucleotide sequence having at least 75% identity (such as at least 80, 85, 90, 91, 92, 93, 94, 95, 96, 97, 98, 99 or 100% identity) to a nucleotide sequence of SEQ ID NO: 24 (shown below). In this regard, conservative substitutions may be useful.

| SEQ ID NO: | Probe sequence (5′ to 3′) |
|---|---|
| 24 | TTA CCG TTA GTG CTG GTT GCC CAC |

An alternative means for defining variant probe sequences is by defining the number of nucleotides that differ between the variant sequence and the reference probe sequence. Thus, in one embodiment, a probe of the present invention comprises (or consists of) a nucleic acid sequence that differs from SEQ ID NO: 24 by no more than 6 nucleotides, such as by no more than 5, 4, 3, 2 or 1 nucleotides. In this regard, conservative substitutions may be useful.

A fragment of the above-mentioned probe sequence may also be employed, wherein the fragment comprises at least 18 consecutive nucleotides of SEQ ID NO: 24. Thus, in one embodiment, a probe of the present invention comprises (or consists of) a fragment of SEQ ID NO: 24 (or sequence variants thereof as defined above), wherein said fragment comprises at least 18, 19, 20, 21, 22 or 23 consecutive nucleotides thereof.

Suitable conventional washing conditions for removing unbound hilA probe are as defined above with respect to the lacZ probe.

In one embodiment, the hilA probe comprises a label or tag. Suitable labels or tags include those defined above with respect to the lacZ probe.

As discussed above with respect to the lacZ probe (detection of S. enterica subsp. IIIa/IIIb) in one embodiment, following hybridisation of labelled/tagged hilA probe to amplification product, the label/tag is associated with the bound amplification product. In one embodiment, the assay comprises detecting the label or capturing the tag (e.g. following separation of unbound probe from the sample) and correlating presence of label or tag with presence of probe bound to amplification product, and hence the presence of S. enterica subsp. I.

Suitable methods for detecting the label or capturing the tag are described above with respect to the lacZ probe (detection of S. enterica subsp. IIIa/IIIb)—e.g. techniques employing a substrate or solid support, such as a membrane or magnetic bead.

The hilA probe may comprise a minor groove binder component.

In one embodiment, the hilA probe comprises reporter and quencher fluorophores (as discussed above with respect to the lacZ probe (detection of S. enterica subsp. IIIa/IIIb)). In one embodiment, cleavage of the hilA probe separates the reporter and quencher fluorophores, which may result in a detectable fluorescent signal, or in a detectable change in a fluorescent signal.

Thus, in one embodiment, the detection step comprises (e.g. after separating unhybridised probe from the sample) cleaving the hybridised probe to separate the reporter and quencher fluorophores; and detecting a fluorescent signal or detecting a change in a fluorescent signal; wherein said fluorescent signal, or change in fluorescent signal, is indicative of the presence of the amplification product, and hence the presence of S. enterica subsp. I.

By way of example, bound probe may be cleaved by an extending polymerase with 5′ to 3′ exonuclease activity, as may occur in a real-time PCR assay, such as a Taqman® assay. In one embodiment, the Taqman® system for amplifying and detecting a target nucleic acid sequence is employed (as described above).

In one aspect, the hilA probe is immobilised onto a support or platform. Suitable supports/platforms are discussed above with respect to the lacZ probe (detection of S. enterica subsp. IIIa/IIIb). Immobilisation to a support/platform may be achieved by a variety of conventional means—e.g. as discussed above with respect to the lacZ probe (detection of S. enterica subsp. IIIa/IIIb).

In one aspect, the hilA amplification product is a double-stranded nucleic acid molecule and is detected by a method comprising melt curve analysis. Melting curve analysis is an assessment of the dissociation characteristics of double-stranded nucleic acid (e.g. DNA) during heating.

In one aspect, the hilA amplification product is detected by a method comprising contacting the sample with an enzyme (such as a restriction endonuclease) that digests the amplification product, and identifying digestion products. In this aspect, the restriction endonuclease recognises a restriction site that is located within the sequence of the amplification product. The presence of digestion products confirms that amplification product is present and hence confirms the presence of S. enterica subsp. I. The absence of digestion products confirms that amplification product is absent, and hence confirms the absence of S. enterica subsp. I.

The digestion products may be detected by any known means, as discussed above with respect to detection of S. enterica subsp. IIIa/IIIb.

In one embodiment, as discussed above, the method comprises contacting the sample with a second pair of forward and reverse oligonucleotide primers, which act as an internal amplification control to confirm presence of Salmonella sp (e.g. the control primers hybridise to the Salmonella ttrRSBCA locus). In one embodiment, as discussed above, detection of the control amplification product confirms the presence of Salmonella sp in the sample.

The sample may be contacted with control primers and/or control probe simultaneously with (in parallel with or in combination with) the forward and reverse primers and/or probe of the invention, or sequentially with (prior to or after) the forward and reverse primers and/or probe of the invention.

The method defined herein enables quantitative estimates of S. enterica subsp. I bacterial load to be determined (e.g. for clinical guidance, determining therapy, patient management or assessing vaccine efficacy). The techniques as described above for measuring the amount of lacZ amplification product are useful for measuring the amount of hilA amplification product and hence quantifying the amount of S. enterica subsp. I nucleic acid in a sample.

In one aspect, the present invention provides an in vitro method for quantitating the bacterial load of S. enterica subsp. I in a sample of interest, comprising: (a) carrying out a detection method according to the present invention on said sample of interest; and (b) carrying out said method on a test sample having a predetermined bacterial load of S. enterica subsp. I; and (c) comparing the amount of amplification product detected from the sample of interest with the amount of amplification product detected from the test sample; and thereby quantitating the bacterial load of S. enterica subsp. I in the sample of interest.

In another aspect, the method of the present invention is useful for determining efficacy of a course of treatment for S. enterica subsp. I infection over a period of time, for example a course of therapy, such as drug or vaccine therapy. Thus, in one embodiment, the invention provides an in vitro method of determining the efficacy of an anti-S. enterica subsp. I therapy (such as an anti-S. enterica subsp. I drug) over the course of a period of therapy, comprising: (a) carrying out a detection method according to the present invention on a first sample obtained at a first time point within or prior to the period of therapy; (b) carrying out said method on one or more samples obtained at one or more later time points within or after the period of therapy; and (c) comparing the amount of amplification product detected from the first sample with the amount of amplification product detected from the one or more later samples; and thereby determining drug efficacy over the course of the period of drug therapy. In one embodiment, a reduction in the quantity of amplification product detected from the one or more later samples, as compared with the quantity of amplification product detected from the first sample, indicates efficacy of the drug against S. enterica subsp. I.

In another aspect, the present invention is useful for determining the efficacy of a vaccine against infection with S. enterica subsp. I. In one embodiment, the present invention provides an in vitro method of determining the efficacy of a vaccine against S. enterica subsp. I, comprising: (a) carrying out a detection method according to the present invention on a first sample obtained from a patient at a first time point prior to vaccination; (b) carrying out said method on a sample obtained from said patient at one or more later time points after vaccination and following challenge with S. enterica subsp. I; and (c) comparing the amount of amplification product detected from the first sample with the amount of amplification product detected from the one or more later samples; and thereby determining vaccine efficacy. In one embodiment, a reduction in the quantity of amplification product detected from the one or more later samples, as compared with the quantity of amplification product detected from the first sample, indicates efficacy of the vaccine against infection with S. enterica subsp. I.

The invention also provides reagents such as forward primers, reverse primers, probes, combinations thereof, and kits comprising said reagents, for use in the above-described methods for detecting S. enterica subsp. I.

In one embodiment, the sequence of the forward primer and/or reverse primer and/or probe does not comprise or consist of the entire nucleic acid sequence of SEQ ID NO: 13 or 14, or the complement thereof.

In one aspect, the invention provides a forward oligonucleotide primer as defined above, which hybridises to a target nucleic acid sequence located within the complement of SEQ ID NO: 13 or 14.

In one embodiment, the forward primer hybridises to a target sequence located between residues 1-1560 of the complement of SEQ ID NO: 13 or 14. Within this range, the forward primer target sequence may be located in a region from residue 200, 400, 600, 800, 1000, 1200, 1300, 1350, 1400, 1450, 1460, 1470, 1480, 1490, 1500, 1505, 1506, 1507, 1508, 1509, 1510 or 1511 of the complement of SEQ ID NO: 13 or 14. Within this range, the forward primer target sequence may be located in a region up to residue 1555, 1550, 1545, 1540, 1539, 1538, 1537, 1536, 1535 or 1534 of the complement of SEQ ID NO: 13 or 14. For example, the forward primer may hybridise to a target sequence located between residues 1475-1555 of the complement of SEQ ID NO: 13 or 14, such as a target sequence located between residues 1500-1550 of the complement of SEQ ID NO: 13 or 14. In one embodiment, the forward primer target sequence is defined by residues 1511-1534 of the complement of SEQ ID NO: 13 or 14.

In one embodiment, said forward primer target nucleic acid sequence comprises (or consists of) a nucleotide sequence that is at least 75% identical to (such as 80, 85, 86, 87, 88, 89, 90, 91, 92, 93, 94, 95, 96, 97, 98, 99 or 100% identical to) SEQ ID NO: 15 or 16, or a fragment thereof as defined above. In one embodiment, said forward primer target nucleic acid sequence is specific to S. enterica subsp. I.

In one embodiment, the forward primer comprises (or consists of) a nucleotide sequence having at least 75% identity to (such as 80, 85, 86, 87, 88, 89, 90, 91, 92, 93, 94, 95, 96, 97, 98, 99 or 100% identity to) a nucleotide sequence of SEQ ID NO: 19 or 20.

In one embodiment, the forward primer comprises (or consists of) a fragment of SEQ ID NO: 19 or 20 (or a sequence variant thereof as defined above) wherein said fragment comprises at least 15 consecutive nucleotides thereof.

In one embodiment, said fragment comprises at least 16, 17, 18, 19, 20, 21, 22 or 23 consecutive nucleotides thereof.

In one aspect, the invention provides a reverse oligonucleotide primer as defined above, which hybridises to a target nucleic acid sequence located within SEQ ID NO: 13 or 14.

In one embodiment, the reverse primer hybridises to a target sequence located between residues 1560-1662 of SEQ ID NO: 13 or 14. Within this range, the forward primer target sequence may be located in a region from residue 1561, 1562, 1563, 1564, 1565, 1566, 1567 or 1568 of SEQ ID NO: 13 or 14. Within this range, the forward primer target sequence may be located in a region up to residue 1660, 1650, 1640, 1630, 1625, 1620, 1615, 1610, 1605, 1600, 1595, 1594, 1593, 1592, 1591 or 1590 of SEQ ID NO: 13 or 14. For example, the reverse primer may hybridise to a target sequence located between residues 1560-1625 of SEQ ID NO: 13 or 14, such as a target sequence located between residues 1565-1600 of SEQ ID NO: 13 or 14. In one embodiment, the reverse primer target sequence is defined by residues 1568-1590 of SEQ ID NO: 13 or 14.

In one embodiment, said reverse primer target nucleic acid sequence comprises (or consists of) a nucleotide sequence that is at least 75% identical to (such as 80, 85, 86, 87, 88, 89, 90, 91, 92, 93, 94, 95, 96, 97, 98, 99 or 100% identical to) a nucleotide sequence of SEQ ID NO: 17 or 18. In one embodiment, said target nucleic acid sequence is specific to S. enterica subsp. I.

In one embodiment, the reverse primer comprises (or consists of) a nucleotide sequence having at least 75% identity to (such as 80, 85, 86, 87, 88, 89, 90, 91, 92, 93, 94, 95, 96, 97, 98, 99 or 100% identity to) a nucleotide sequence of SEQ ID NO: 21 or 22.

In one embodiment, the reverse primer comprises (or consists of) a fragment of SEQ ID NO: 21 or 22 (or a sequence variant thereof as defined above) wherein said fragment comprises at least 15 consecutive nucleotides thereof.

In one embodiment, said fragment comprises at least 16, 17, 18, 19, 20, 21 or 22 consecutive nucleotides thereof.

In one embodiment, the forward primer and/or the reverse primer comprise a tag or label, as described above.

The present invention further provides a pair of forward and reverse oligonucleotide primers, comprising a forward primer as defined above and a reverse primer as defined above.

The present invention also provides a probe, such as an oligonucleotide probe as defined above. In one embodiment, the probe hybridises to a target binding sequence located within the complement of SEQ ID NO: 13 or 14.

In one embodiment, the probe binds a target nucleotide sequence located between residues 1521-1580 of the complement of SEQ ID NO: 13 or 14. Within this range, the probe target sequence may be located in a region from residue 1525, 1530, 1531, 1532, 1533, 1534, 1535, 1536 or 1537 of the complement of SEQ ID NO: 13 or 14. Within this range, the probe target sequence may be located in a region up to residue 1575, 1570, 1569, 1568, 1567, 1566, 1565, 1564, 1563, 1562, 1561 or 1560 of the complement of SEQ ID NO: 13 or 14. For example, the forward primer may hybridise to a target sequence located between residues 1530-1570 of the complement of SEQ ID NO: 13 or 14, such as a target sequence located between residues 1535-1565 of the complement of SEQ ID NO: 13 or 14. In one embodiment, the forward primer target sequence is defined by residues 1537-1560 of the complement of SEQ ID NO: 13 or 14.

In one embodiment, the target binding sequence for the probe comprises (or consists of) a target sequence that is at least 75% identical (such as at least 80, 85, 90, 91, 92, 93, 94, 95, 96, 97, 98, 99 or 100% identical) to a nucleotide sequence of SEQ ID NO: 23, or to a fragment thereof having at least 19 consecutive nucleotides thereof (such as at least 20, 21, 22 or 23 consecutive nucleotides thereof).

In one embodiment, said probe comprises (or consists of) a nucleotide sequence having at least 75% identity to (such as at least 80, 85, 86, 87, 88, 89, 90, 91, 92, 93, 94, 95, 96, 97, 98, 99 or 100% identity to) a nucleotide sequence of SEQ ID NO: 24, or a fragment thereof having at least 18 consecutive nucleotides thereof (such as at least 19, 20, 21, 22 or 23 consecutive nucleotides thereof).

In one embodiment, the probe comprises a tag or label, as described above, or reporter and quencher fluorophores, as described above.

The present invention also provides a kit for detecting S. enterica subsp. I bacteria in a sample, comprising a pair of forward and reverse oligonucleotide primers as defined above. The kit optionally comprises amplification reagents such as a polymerase (e.g. a polymerase having 5′-3′ exonuclease activity such as Taq polymerase) and/or DNA precursors.

The kit optionally comprises reagents for detection of the amplification product. In one embodiment, reagents for detection of the amplification product comprise an oligonucleotide probe as described above, which hybridises to said amplification product. In one embodiment, reagents for detection of the amplification product comprise an enzyme such as a restriction endonuclease (such as HhaI) that digests the amplification product, as described above.

In one embodiment, the above-described method for detecting S. enterica subsp. I further comprises detecting S. enterica subsp. IIIa and/or IIIb. By way of example, S. enterica subsp. IIIa and/or IIIb may be detected using the method described herein.

In one embodiment, the above-described method for detecting S. enterica subsp. IIIa and/or IIIb further comprises detecting S. enterica subsp. I. By way of example, S. enterica subsp. I may be detected using the method described herein.

Accordingly, in one aspect, the invention provides a method for detecting S. enterica subsp. I and S. enterica subsp. IIIa and/or IIIb.

In one embodiment, the method for detecting S. enterica subsp. IIIa and/or IIIb comprises:
(a) contacting the sample with a pair of forward and reverse oligonucleotide primers, wherein said forward and reverse primers hybridise to target nucleic acid sequences located within the lacZ gene of S. enterica subsp. III, or the complement thereof;
(b) extending said forward and reverse primers to generate an amplification product; and
(c) detecting the amplification product.

In one embodiment, the method for detecting S. enterica subsp. I comprises:
(a) contacting the sample with a pair of forward and reverse oligonucleotide primers, wherein said forward and reverse primers hybridise to target nucleic acid sequences located within the hilA gene of S. enterica subsp. I, or the complement thereof;
(b) extending said forward and reverse primers to generate an amplification product; and
(c) detecting the amplification product.

Accordingly, in one embodiment, the invention provides an assay for identifying S. enterica subsp. I and S. enterica subsp. IIIa/IIIb in a sample. In one embodiment, this assay enables S. enterica subsp. I and S. enterica subsp. III to be identified, and distinguished, in approximately two hours. In contrast, using conventional phenotypic methods, it currently takes several days (and in some cases as long as 28 days) to identify and distinguish Salmonella subsp.

In one embodiment, the method for detecting S. enterica subsp. I and the method for detecting S. enterica subsp. IIIa and/or IIIb are carried out substantially simultaneously, for example in parallel (e.g. in separate reactions at substantially the same time) or in combination (e.g. in the same reaction at substantially the same time).

In one embodiment, the invention provides an assay for simultaneously detecting S. enterica subsp. I and S. enterica subsp. IIIa and/or IIIb in a sample, the method comprising:
(a) contacting the sample with:
   (i) a pair of forward and reverse oligonucleotide primers, wherein said forward and reverse oligonucleotide primers hybridise to target nucleic acid sequences located within the lacZ gene of S. enterica subsp. III, or the complement thereof; and
   (ii) a pair of forward and reverse oligonucleotide primers, wherein said forward and reverse oligonucleotide primers hybridise to target nucleic acid sequences located within the hilA gene of S. enterica subsp. I, or the complement thereof;
(b) extending said hilA forward and reverse oligonucleotide primers and said lacZ forward and reverse oligonucleotide primers to generate a hilA amplification product and a lacZ amplification product, respectively; and
(c) detecting said hilA amplification product and said lacZ amplification product.

The forward and reverse lacZ primers may be used in the same reaction as the forward and reverse hilA primers (i.e. detection of S. enterica subsp. III and S. enterica subsp. I is carried out simultaneously and in combination).

Alternatively, the sample may be divided between at least two reactions (i.e. at least a first and second reaction), wherein the forward and reverse lacZ primers may be used in a first reaction and the forward and reverse hilA primers may be used in a second reaction (i.e. detection of S. enterica subsp. III and S. enterica subsp. I is carried out simultaneously and in parallel).

In one embodiment, the method for detecting S. enterica subsp. I and the method for detecting S. enterica subsp. IIIa and/or IIIb are carried out sequentially. By way of example, the method for detecting S. enterica subsp. I is performed on a sample, and then the method for detecting S. enterica subsp. IIIa and/or IIIb is carried out on the sample. Alternatively, the method for detecting S. enterica subsp. IIIa and/or IIIb may be performed on a sample, and then the method for detecting S. enterica subsp. I is carried out on the sample.

In one embodiment, the invention provides an assay for sequentially detecting S. enterica subsp. I and S. enterica subsp. IIIa and/or IIIb in a sample, the method comprising:
(a) contacting the sample with a pair of forward and reverse oligonucleotide primers, wherein said forward and reverse oligonucleotide primers hybridise to target nucleic acid sequences located within the lacZ gene of S. enterica subsp. III, or the complement thereof;
(b) extending said lacZ forward and reverse oligonucleotide primers to generate a lacZ amplification product; and
(c) detecting said amplification product;
   and then:
(d) contacting the sample with a pair of forward and reverse oligonucleotide primers, wherein said forward and reverse oligonucleotide primers hybridise to target nucleic acid sequences located within the hilA gene of S. enterica subsp. I, or the complement thereof;
(e) extending said hilA forward and reverse oligonucleotide primers to generate an amplification product; and
(f) detecting said hilA amplification product.

In an alternative embodiment, steps (d)-(f) above are performed prior to carrying out steps (a)-(c) above.

In an alternative embodiment, steps (a)-(b) above are performed, and then steps (d)-(f) are performed, and then detection steps (c) and (f) are performed. In a further alternative embodiment, steps (d)-(f) above are performed, and then steps (a)-(b) above are performed, and then detection steps (c) and (f) are performed.

The invention also provides an in vitro method for quantitating S. enterica subsp. I bacterial load and S. enterica subsp. IIIa/and IIIb bacterial load in a sample of interest, comprising: quantitating S. enterica subsp. I bacterial load in the sample by a method as described above; and further comprising quantitating S. enterica subsp. IIIa and/or IIIb bacterial load in the sample. In one embodiment, the method for quantitating S. enterica subsp. IIIa and/or IIIb bacterial load in the sample is as described above.

The invention also provides an in vitro method for quantitating S. enterica subsp. I bacterial load and S. enterica subsp. IIIa/and IIIb bacterial load in a sample of interest, comprising: quantitating S. enterica subsp. I bacterial load in the sample; and further comprising quantitating S. enterica subsp. IIIa and/or IIIb bacterial load in the sample by a method described above. In one embodiment, the method for quantitating S. enterica subsp. I bacterial load in the sample is as described above.

The invention also provides an in vitro method of determining the efficacy of a drug against S. enterica subsp. I and S. enterica subsp. IIIa/b over the course of a period of therapy, comprising: determining efficacy of the drug against S. enterica subsp. I by a method described above; and further comprising determining efficacy of the drug against S. enterica subsp. IIIa/b. In one embodiment, the method for determining efficacy of the drug against anti-S. enterica subsp. IIIa/b is as described above.

The invention also provides an in vitro method of determining the efficacy of a drug against S. enterica subsp. I and S. enterica subsp. IIIa/b over the course of a period of therapy, comprising: determining efficacy of the drug against S. enterica subsp. I; and further comprising determining efficacy of the drug against S. enterica subsp. IIIa/b by a method described above. In one embodiment, the method for determining efficacy of the drug against S. enterica subsp. I is as described above.

The invention also provides an in vitro method of determining the efficacy of a vaccine against S. enterica subsp. I infection and S. enterica subsp. IIIa/b infection, comprising: determining efficacy of the vaccine against S. enterica subsp. I by a method described above; and further comprising determining efficacy of the vaccine against S. enterica subsp. IIIa/b. In one embodiment, the method for determining efficacy of the vaccine against anti-S. enterica subsp. IIIa/b is as described above.

The invention also provides an in vitro method of determining the efficacy of a vaccine against S. enterica subsp. I and S. enterica subsp. IIIa/b over the course of a period of therapy, comprising: determining efficacy of the vaccine against S. enterica subsp. I; and further comprising determining efficacy of the vaccine against S. enterica subsp. IIIa/b by a method described above. In one embodiment, the method for determining efficacy of the vaccine against S. enterica subsp. I is as described above.

In one embodiment, for detection of the S. enterica subsp. I and S. enterica subsp. IIIa/b in the same reaction (at the same time or sequentially), the fluorophores attached to the subsp. I- and subsp. 3-specific probes are different.

The invention also provides a set of primers for detecting S. enterica subsp. I and S. enterica subsp. IIIa and/or IIIb in a sample.

The set of primers comprises a forward and/or a reverse primer for detecting S. enterica subsp. I (e.g. as described above). In one embodiment, the set of primers comprises forward and/or reverse hilA primers for detecting S. enterica subsp. I as described above. The set of primers also comprises a forward and/or a reverse primer for detecting S. enterica subsp. IIIa and/or IIIb (e.g. as described above). In one embodiment, the set of primers comprises forward and/or reverse lacZ primers for detecting S. enterica subsp. IIIa and/or IIIb as described above.

In one embodiment, the invention provides a set of primers for detecting S. enterica subsp. I and S. enterica subsp. IIIa and/or IIIb in a sample, the set of primers comprising:
(i) one or more primers for detecting S. enterica subsp. I, for example, one or more hilA primers, such as one or more primers selected from:
(a) a forward hilA primer that hybridises to a target nucleic acid sequence, which target nucleic acid sequence comprises a nucleotide sequence that is at least 75% identical to SEQ ID NO: 15 or 16, or a fragment thereof having at least 15 consecutive nucleotides thereof; and
(b) a reverse hilA primer that hybridises to a target nucleic acid sequence, which target nucleic acid sequence comprises a nucleotide sequence that is at least 75% identical to a nucleotide sequence of SEQ ID NO: 17 or 18, or a fragment thereof having at least 15 consecutive nucleotides thereof;
and
(ii) one or more primers for detecting S. enterica subsp. IIIa/IIIb, for example, one or more lacZ primers, such as one or more primers selected from:
(c) a forward lacZ primer that hybridises to a target nucleic acid sequence, which target nucleic acid sequence comprises a nucleotide sequence that is at least 75% identical to SEQ ID NO: 2, or a fragment thereof having at least 15 consecutive nucleotides thereof; and
(d) a reverse lacZ primer that hybridises to a target nucleic acid sequence, which target nucleic acid sequence comprises a nucleotide sequence that is at least 75% identical to a nucleotide sequence of SEQ ID NO: 3, or a fragment thereof having at least 15 consecutive nucleotides thereof.

Thus, in one embodiment, the invention provides a set of primers for detecting S. enterica subsp. I and S. enterica subsp. IIIa and/or IIIb in a sample, the set of primers comprising:
(a) a forward hilA primer that hybridises to a target nucleic acid sequence, which target nucleic acid sequence comprises a nucleotide sequence that is at least 75% identical to SEQ ID NO: 15 or 16, or a fragment thereof having at least 15 consecutive nucleotides thereof;
(b) a reverse hilA primer that hybridises to a target nucleic acid sequence, which target nucleic acid sequence comprises a nucleotide sequence that is at least 75% identical to a nucleotide sequence of SEQ ID NO: 17 or 18, or a fragment thereof having at least 15 consecutive nucleotides thereof;
(c) a forward lacZ primer that hybridises to a target nucleic acid sequence, which target nucleic acid sequence comprises a nucleotide sequence that is at least 75% identical to SEQ ID NO: 2, or a fragment thereof having at least 15 consecutive nucleotides thereof; and
(d) a reverse lacZ primer that hybridises to a target nucleic acid sequence, which target nucleic acid sequence comprises a nucleotide sequence that is at least 75% identical to a nucleotide sequence of SEQ ID NO: 3, or a fragment thereof having at least 15 consecutive nucleotides thereof.

The invention also provides a set of oligonucleotide probes for detecting S. enterica subsp. I and S. enterica subsp. IIIa and/or IIIb in a sample.

The set of probes comprises a probe for detecting S. enterica subsp. I (e.g. as described above). The set of probes also comprises a probe for detecting S. enterica subsp. IIIa and/or IIIb (e.g. as described above). In one embodiment, the set of probes comprises a hilA probe for detecting S. enterica subsp. I—e.g. as described above. In one embodiment, the set of probes comprises a lacZ probe for detecting S. enterica subsp. IIIa and/or IIIb—e.g. as described above.

Thus, in one embodiment, the invention provides a set of probes for detecting S. enterica subsp. I and S. enterica subsp. IIIa and/or IIIb in a sample, the set of probes comprising:
(a) a hilA probe that hybridises to a target nucleic acid sequence, which target nucleic acid sequence comprises a nucleotide sequence that is at least 75% identical to a nucleotide sequence of SEQ ID NO: 23, or a fragment thereof having at least 18 consecutive nucleotides thereof; and/or
(b) a lacZ probe that hybridises to a target nucleic acid sequence, which target nucleic acid sequence comprises a nucleotide sequence that is at least 75% identical to a nucleotide sequence of SEQ ID NO: 6, or a fragment thereof having at least 18 consecutive nucleotides thereof.

In one embodiment, the set of probes comprises:
(a) a hilA probe that comprises a nucleotide sequence having at least 75% identity to a nucleotide sequence of SEQ ID NO: 24, or a fragment thereof comprising at least 18 consecutive nucleotides thereof; and/or
(b) a lacZ probe that comprises a nucleotide sequence having at least 75% identity to a nucleotide sequence of SEQ ID NO: 7, or a fragment thereof comprising at least 18 consecutive nucleotides thereof.

The invention also provides a kit for detecting S. enterica subsp. I and S. enterica subsp. IIIa and/or IIIb in a sample.

The kit comprises a set of primers for detecting S. enterica subsp. I and S. enterica subsp. IIIa and/or IIIb. In one embodiment, the set of primers for detecting S. enterica subsp. I is as discussed above. In one embodiment, the set of primers for detecting S. enterica subsp. IIIa and/or IIIb is as discussed above.

The kit also comprises reagents for amplification of a S. enterica subsp. I—specific nucleic acid sequence and a S. enterica subsp. IIIa and/or IIIb—specific nucleic acid sequence; and/or reagents for detection of the amplification products.

The reagents for detection of the amplification products optionally comprise oligonucleotide probes for detecting the S. enterica subsp. I and/or S. enterica subsp. IIIa and/or III amplification products. In one embodiment, the set of probes for detecting the S. enterica subsp. amplification product is as defined above. In one embodiment, the set of probes for detecting the S. enterica subsp. IIIa and/or IIIb amplification product is as discussed above.

All the embodiments described above with respect to the methods and reagents for detecting S. enterica subsp. IIIa and/or IIIb in a sample apply equally to the above-described methods for detecting S. enterica subsp. I and S. enterica subsp. IIIa and/or IIIb in a sample.

All the embodiments described above with respect to the methods and reagents for detecting S. enterica subsp. I in a sample apply equally to the above-described methods for detecting S. enterica subsp. I and S. enterica subsp. IIIa and/or IIIb in a sample.

The present invention is discussed in more detail by means of the Examples described below, and by the Figures.

FIG. 1 illustrates the four main steps of a conventional real-time Taqman assay, labelled I-IV, namely (I) “Polymerisation”, (II) “Strand displacement” (III) “Cleavage” and (IV) “Polymerisation completed”.

Forward primer (4), reverse primer (5) and probe (6) are mixed with a buffered solution comprising sample DNA, dNTPs and a thermostable DNA polymerase enzyme (not shown), and added to the reaction vessel. The target dsDNA (1) (if present) is then denatured by heating to a temperature above its Tm, causing strand separation.

As illustrated in Step (I) of FIG. 1, “Polymerisation”, the temperature is then lowered sufficiently for hybridisation to occur between forward primer (4) and complementary, non-coding/anti-sense strand (3); between reverse primer (5) and coding/sense strand (2); and between probe (6) and either strand (2) or (3). In the particular embodiment illustrated in FIG. 1, step (I), probe (6) is bound to complementary, non-coding/anti-sense strand (3). In this particular example, probe (6) is labelled at its 5′ end with a reporter fluorophore (R) and at its 3′ end with a quencher fluorophore (Q).

DNA synthesis then proceeds by extension of the bound forward and reverse primers by DNA polymerase, generating extending strands (7) and (8).

As illustrated in step (II) of FIG. 1 “Strand displacement”, bound probe (6) obstructs the progress of one of the extending strands (extending strand (7) in this particular example). This obstruction is then circumvented by the DNA Taq polymerase, which possesses 5′ to 3′ exonuclease activity, and hence can enzymatically degrade probe (6).

Step (III), “Cleavage” illustrates that as probe (6) is cleaved the two fluorophores (R) and (Q) present on probe (6) are separated, which alters the relative fluorescent signal.

Step (IV), “Polymerisation completed” shows complete extension of extending strands (7) and (8) along the length of coding/sense strand (2) and complementary, non-coding/anti-sense strand (3), and degradation of probe (6). The first round of PCR thermal cycling is hence complete.

On each successive round of PCR thermal cycling, probe (6) is cleaved and fluorophores (R) and (Q) are separated. The resulting fluorescent signal is cumulative and increases exponentially during PCR amplification.

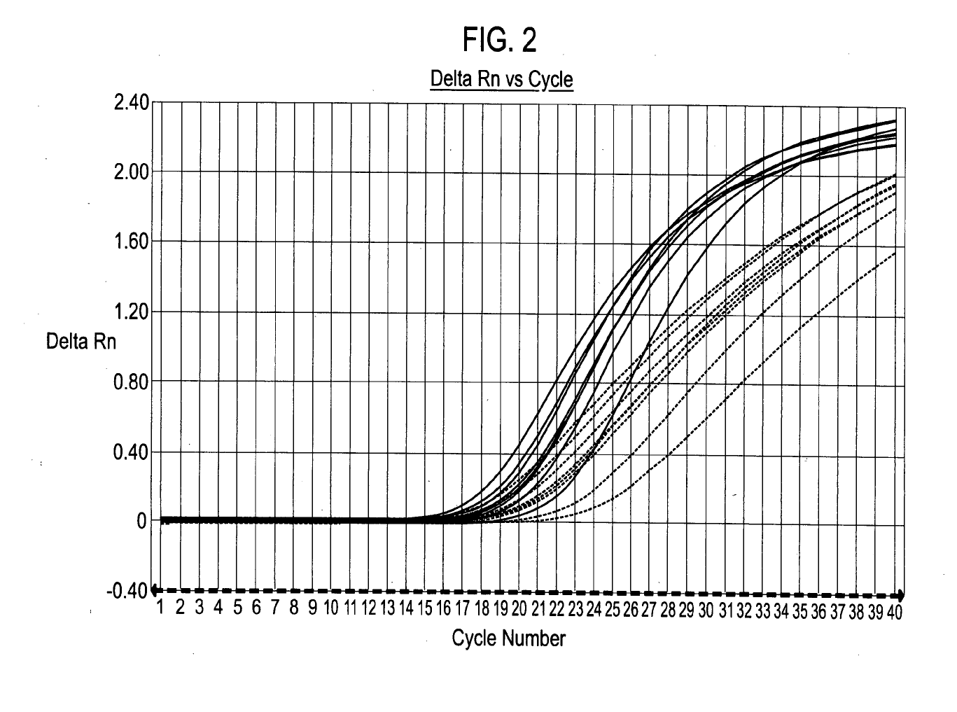

FIG. 2 illustrates Real-time detection of Salmonella enterica and identification of subspecies arizonae and diarizonae. The cycle number (1-40) is plotted versus the Delta Rn. The Delta Rn represents the Rn (the increase in the fluorescence signal of the reporter fluorochrome relative to the internal standard dye) minus the baseline signal established in the early PCR cycles. Results for the Salmonella genus-specific target ttr are indicated X, those for the arizonae and diarizonae-specific target are indicated Y.

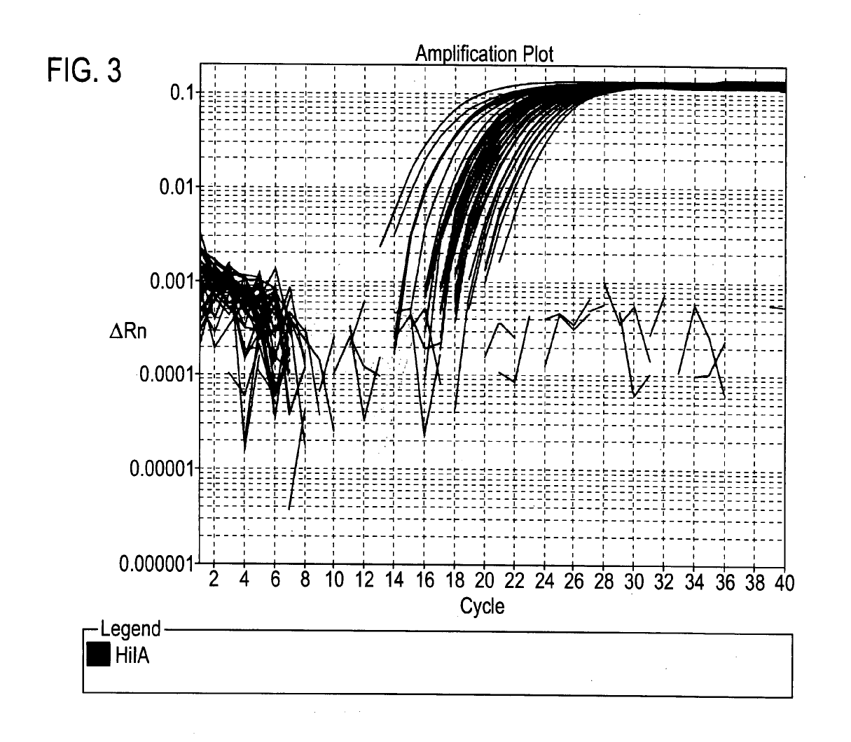

FIG. 3 illustrates Real-time detection of Salmonella enterica subspecies enterica. The cycle number (1-40) is plotted versus the Delta Rn (the increase in the fluorescence signal of the reporter fluorochrome relative to the internal standard dye minus the baseline signal).

### KEY TO SEQ ID NOS

SEQ ID NO: 1 lacZ gene of S. enterica subsp. diarizonae (IIIb) Accession number AY746956.
SEQ ID NO: 2 lacZ target site for forward primer
SEQ ID NO: 3 lacZ target site for reverse primer
SEQ ID NO: 4 sequence of lacZ forward primer
SEQ ID NO: 5 sequence of lacZ reverse primer
SEQ ID NO: 6 lacZ target site for probe
SEQ ID NO: 7 sequence of lacZ probe
SEQ ID NO: 8 control forward primer
SEQ ID NO: 9 control reverse primer
SEQ ID NO: 10 control probe
SEQ ID NO: 11 lacZ gene of S. enterica subsp. diarizonae (IIIb) provided by the University of Washington
SEQ ID NO: 12 lacZ gene of S. enterica subsp. arizonae (IIIa) provided by the University of Washington
SEQ ID NO: 13 hilA gene of S. enterica serovar Typhimurium LT2 (nt. 8999-10660 of GenBank accession number AE008831)
SEQ ID NO: 14 hilA gene of S. enterica serovar Typhimurium (nt. 847-2508 of GenBank accession number 025352)
SEQ ID NO: 15 hilA target site for forward primer
SEQ ID NO: 16 hilA target site for forward primer
SEQ ID NO: 17 hilA target site for reverse primer
SEQ ID NO: 18 hilA target site for reverse primer
SEQ ID NO: 19 sequence of hilA forward primer
SEQ ID NO: 20 sequence of hilA forward primer
SEQ ID NO: 21 sequence of hilA reverse primer
SEQ ID NO: 22 sequence of hilA reverse primer
SEQ ID NO: 23 hilA target site for probe
SEQ ID NO: 24 sequence of hilA probe

### EXAMPLES

### Example 1

### Real Time PCR Assay for Salmonella enterica Subspecies III

Real-time PCR can be used for rapid and accurate detection of pathogens, therefore we sought to develop and validate a novel duplex 5′ nuclease (TaqMan®) real-time PCR for the rapid and reliable identification of S. enterica subsp. arizonae (IIIa) and diarizonae (IIIb).

### Method:

### Overview:

A primer/TaqMan® probe set was designed to target a gene sequence specific to S. enterica subsp. Ill. As an internal amplification control to confirm presence of Salmonella sp., a second primer/TaqMan® probe set was used, which simultaneously detects the Salmonella-specific ttrRSBCA locus.

The assay was validated on an Applied Biosystems 7500 Real-Time PCR System using a panel of 166 S. arizonae and diarizonae, 37 Salmonella belonging to subspecies I, II and IV-VI, and 34 isolates of other enterobacterial species (all previously identified by serology and/or biochemistry).

### Bacterial Strains:

73 S. arizonae and 93 diarizonae from human infections, reptiles, food for consumption by humans and pets, and of unknown origin isolated between January 2004 and August 2007, and previously identified using biochemistry and Kauffmann-White serology were used in this study. The following organisms were used as negative controls: 37 Salmonella belonging to subspecies I, II, IV, V and VI, and 34 isolates of other enterobacterial species (Hafnia, Citrobacter, Enterobacter, Escherichia or Proteus spp.).

### DNA Template Preparation:

Boiled cell lysates were prepared by emulsifying one colony in 8-strip PCR tubes containing 100 μl of sterile distilled water and boiling for 10 minutes. Lysates were stored at −20° C.

### Primer and TaqMan® Probe Design:

The sequences for the Arizona-specific primer/TaqMan® probe set were designed based on the S. arizonae lacZ sequence (GenBank accession number AY746956) with respect to guidelines from Applied Biosystems using Primer3 software (http://frodo.wi.mit.edu/cgi-bin/primer3/primer3_www.cgi). Specificity of the sequences was tested by BLASTn search.

A second primer/TaqMan® probe set targeted to the ttrRSBCA locus (required for tetrathionate respiration) and shown previously to be suitable for detection of Salmonella were utilised to act as an internal amplification control and confirm presence of Salmonella DNA.

| Primer/Probe name | Sequence |
|---|---|
| IacZ-F2 | 5′ GCAAAACCTACCGGATTGAT 3′ |
| (SEQ ID NO: 4) | |
| IacZ-R2 | 5′ TCCACTTACTTTCCCACCTC 3′ |
| (SEQ ID NO: 5) | |
| ArizLZPr | 5′ Yakima Yellow- |
| (SEQ ID NO: 7) | CATGGCGAAATGCAGATCGACATC- |
| | Black Hole Quencher-1 3′ |
| Control Primer/ | |
| Probe name | Sequence |
| ttr-6 forward | 5′ CTCACCAGGAGATTACAACATGG 3′ |
| (SEQ ID NO: 8) | |
| ttr-4 reverse | 5′ AGCTCAGACCAAAAGTGACCATC 3′ |
| (SEQ ID NO: 9) | |
| ttr-5 probe | 5′ FAM- |
| (SEQ ID NO: 10) | CACCGACGGCGAGACCGACTTT- |
| | Black Hole Quencher-1 3′ |

### Real-Time PCR Assays:

The assay was validated in-house on an ABI Prism 7500 Real-Time PCR System (Applied Biosystems). Optimised reactions (25 μl volume) contained 1×qPCR Mastermix Plus Low ROX (Eurogentec), 400 nM each of ttr-6 and ttr-4, 50 nM ttr-5, 900 nM each of lacZ-F2 and lacZ-R2, 200 nM ArizLZPr and 2.5 μl of boiled cell lysate. PCR products were detected directly by the TaqMan® machine monitoring the increase in fluorescence where a numerical value, the CT value (threshold cycle), was assigned.

### Results:

The assay showed 100% specificity and 99% sensitivity. All Salmonella sp. were positive for ttrRSBCA and 165/166 S. arizonae and diarizonae were positive for the S. arizonae-specific target.

There was no amplification of either target from any Hafnia, Citrobacter, Enterobacter, Escherichia or Proteus spp. The average threshold cycle numbers (CT) were 20 for ttrRSBCA and 22 for the S. arizonae-specific target.

| | | ttr | |
|---|---|---|---|
| Subspecies | Serotype (No. of isolates) | locus | lacZ |
| I (enterica) | Agona (1), Anatum (1), Braenderup (1), | + | − |
| | Chailey (1), Dublin (1), Enteritidis (2), | | |
| | Infantis (1), Muenster (1), Newport (1), | | |
| | Typhimurium (2) and Virchow (1) | | |
| II (salamae) | Artis (1), Bloemfontein (2), Dar-es-salaam | + | − |
| | (2), Degania (1) Locarno (1), Hagenbeck (1), | | |
| | Nairobi (1), Tranaroa (1), Uphill (1) and | | |
| | Un-named (3) | | |
| IIIa | 73 | + | + |
| (arizonae) | | | |
| IIIb | 93 | + | + |
| (diarizonae) | | | |
| IV | Chameleon (1), Houten (2), Seminole (1) | + | − |
| (houtenae) | and Un-named (1) | | |
| V (S. | Malawi (1), Un-named (1), | + | − |
| bongori) | | | |
| VI (indica) | Vrindaban (1) and Un-named (2) | + | − |
| Citrobacter | 10 | − | − |
| Enterobacter | 2 | − | − |
| Escherichia | 7 | − | − |
| Hafnia | 9 | − | − |
| Proteus | 6 | − | − |

In contrast to conventional methods for identification of Salmonella sp., which are laborious and time-consuming (an average minimum turnaround time for full biochemical and serological identification of 14-28 days), the assay described above enabled molecular subspecies identification in less than 2 hours.

### Example 2

### Real Time PCR Assay for Salmonella enterica Subspecies I

A similar approach was used to develop a duplex 5′ nuclease (TaqMan®) real-time PCR for the rapid and reliable identification of S. enterica subsp. I, which constitute the majority of salmonella strains that cause infections in humans.

### Method:

### Overview:

A primer/TaqMan® probe set was designed to target a gene sequence (HilA) specific to S. enterica subsp. I. The assay was initially validated on an Applied Biosystems 7500 Real-Time PCR System using a panel of 109 control Salmonella strains: 66 belonging to subspecies 1 and 43 belonging to subspecies II, Ill, IV, V & VI (all previously identified by serology and/or biochemistry). A further 1009 samples received by the HPA Salmonella Reference Unit were also examined in real time.

### Bacterial Strains:

The study used a total of 1118 controls and samples submitted to HPA Salmonella Reference Unit from humans, animals, infections, food and pets, and the environment and identified using biochemistry and Kauffmann-White serology.

### DNA Template Preparation:

Boiled cell lysates were prepared by emulsifying one colony in 8-strip PCR tubes containing 100 μl of sterile distilled water and boiling for 10 minutes. Lysates were stored at −20° C.

### Primer and TaqMan® Probe Design:

Sequences for the S. enterica subspecies I-specific primer/TaqMan® probe set were designed based on work done in an extended analysis of HilA sequence data available in the public domain (i.e. GenBank) and from DNA sequencing preformed in our laboratory. Specificity of the sequences was tested by BLASTn search.

| Primer/Probe name | Sequence |
|---|---|
| HilA-F | 5′ AGC GTA TWG ATA ATA |
| (SEQ ID NO: 19/20) | ATC CGG GAT 3′ |
| HilA-R | 5′ RTT CCA CAT TTT CTC |
| (SEQ ID NO: 21/22) | GGC AAT AG 3′ |
| HilA Probe | 5′ Cy3- |
| (SEQ ID NO: 24) | TTA CCG TTA GTG CTG GTT |
| | GCC CAC-BHQ2 3′ |

### Real-Time PCR Assays:

The assay was validated in-house on an ABI Prism 7500 Real-Time PCR System (Applied Biosystems). Optimised reactions (25 μl volume) contained 1×qPCR Mastermix Plus Low ROX (Eurogentec), 400 nM each of Hi/A-F and Hi/A-R, 200 nM HilA Probe and 2.5 μl of boiled cell lysate. PCR products were detected directly by the TaqMan® machine monitoring the increase in fluorescence where a numerical value, the CT value (threshold cycle), was assigned.

### Results:

All of the 109 control Salmonella strains were correctly identified: the 66 belonging to subspecies I were all positive by the assay and the 43 belonging to subspecies II, Ill, IV, V & VI were all negative. In the real time study of 1009 samples received by the HPA Salmonella Reference Unit: 68 samples were shown (by biochemistry & serology) to be not Salmonella (these were 19 Citrobacter, 2 Escherichia, 20 Hafnia, 5 unspecified enterobacterial species), or S. enterica of subspecies other than 1 (3 subsp. II, 5 subsp. IIIa, 7 subsp. IIIb and 7 subsp. IV)—all were negative by the subspecies I-specific HilA assay. The remaining 941 isolates were identified as S. enterica subsp. I by biochemistry & serology and consisted of representatives from 157 distinct serotypes including: 141 S. Typhimurium; 84 S. Enteritidis; 55 S. Virchow, 45 S. Newport; 35 S. Kentucky 31 S. Infantis; 30 S. Agona; 22 S. Stanley and 15 S. Bareilly (full list available if required)—of these 938 were positive by the subspecies I-specific HilA assay. The remaining 3 samples (1 S. Infantis, 1 S. Bareilly and 1 S. Unnamed 4, 12:b) were negative.

Altogether 1118 controls & samples were examined: Of these 111 non-S. enterica subsp. I isolates were all negative by the assay; while 1004 of the 1,007 S. enterica subsp. I isolates examined were positive by the assay. In this study the S. enterica subsp. I-specific HilA assay therefore shows 100% specificity and 99.7% specificity.

### Example 3

### Proof of Principle Experiment for a Combination Assay with Both Subsp. I and Subsp. III Primer/Probe Sets

In a brief proof of principle experiment the S. enterica subsp. I-specific and subsp. III-specific primer/probe sets were combined into a single assay

### Bacterial Strains:

A total of 8 S. enterica subsp: I, 4 S. enterica subsp. IIIa, and 4 S. enterica subsp. IIIb controls drawn from the previous studies were tested together with a further 8 Salmonella controls from the other species/subspecies.

### Real-Time PCR Assays:

The assay was validated in-house on an ABI Prism 7500 Real-Time PCR System (Applied Biosystems). Optimised reactions (25 μl volume) contained 1×qPCR Mastermix Plus Low ROX (Eurogentec), 400 nM each of HilA-F and HilA-R, 200 nM HilA Probe, 900 nM each of lacZ-F2 and lacZ-R2, 200 nM ArizLZPr and 2.5 μl of boiled cell lysate. PCR products were detected directly by the TaqMan® machine monitoring the increase in fluorescence where a numerical value, the CT value (threshold cycle), was assigned.

### Results:

The assays in combination performed in a comparable manner to how they performed alone. The eight S. enterica subsp. I controls were positive by the S. enterica subsp. I-specific HilA assay only; while the S. enterica subsp. III controls were positive by the S. enterica subsp. III-specific LacZ assay only. The non-subsp. I/III Salmonella controls were negative by both assay components.

1-83. (canceled)

84. A method for detecting S. enterica subsp. I in a sample, the method comprising:
(a) contacting the sample with a pair of forward and reverse oligonucleotide primers, wherein said forward and reverse primers hybridise to target nucleic acid sequences located within the hilA gene of S. enterica subsp. I, or the complement thereof;
(b) extending said forward and reverse primers to generate an amplification product; and
(c) detecting the amplification product;
wherein the nucleotide sequence of the hilA gene of S. enterica subsp. I has at least 95% identity to the nucleotide sequence of SEQ ID NO: 13 or 14; and
wherein:
(i) the forward primer hybridises to a target nucleic acid sequence located between residues 1200-1560 of the complement of SEQ ID NO: 13 or 14; and/or
(ii) the reverse primer hybridises to a target nucleic acid sequence located between residues 1560-1662 of SEQ ID NO: 13 or 14.

85. A method according to claim 84, wherein said forward primer hybridises to a target nucleic acid sequence located between residues 1475-1555 of the complement of SEQ ID NO: 13 or 14; and/or
wherein said reverse primer hybridises to a target nucleic acid sequence located between residues 1560-1625 of SEQ ID NO: 13 or 14.

86. A method according to claim 84, wherein said forward primer hybridises to a target nucleic acid sequence comprising a nucleotide sequence that is at least 75% identical to SEQ ID NO: 15 or 16, or a fragment thereof having at least 15 consecutive nucleotides thereof; and/or
wherein said reverse primer hybridises to a target nucleic acid sequence comprising a nucleotide sequence that is at least 75% identical to SEQ ID NO: 17 or 18, or a fragment thereof having at least 15 consecutive nucleotides thereof.

87. A method according to claim 84, wherein said forward primer comprises a nucleotide sequence having at least 75% identity to a nucleotide sequence of SEQ ID NO: 19 or 20, or a fragment thereof comprising at least 15 consecutive nucleotides thereof.

88. A method according to claim 84, wherein said reverse primer comprises a nucleotide sequence having at least 75% identity to a nucleotide sequence of SEQ ID NO: 21 or 22, or a fragment thereof comprising at least 15 consecutive nucleotides thereof.

89. A method according to claim 84, wherein said forward primer and/or said reverse primer comprises a tag or label; or wherein a tag or label is incorporated into the amplification product during the primer extension step, and wherein the amplification product is detected by a method comprising capturing the tag or detecting the label.

90. A method according to claim 84, wherein the amplification product is detected by a method comprising:
(i) contacting the sample with an oligonucleotide probe that forms a hybridisation complex with the amplification product, if present; and
(ii) detecting the hybridisation complex.

91. A method according to claim 90, wherein the oligonucleotide probe hybridises to a target nucleic acid sequence comprising a nucleotide sequence that is at least 75% identical to SEQ ID NO: 23, or a fragment thereof having at least 18 consecutive nucleotides thereof.

92. A method according to claim 90, wherein the oligonucleotide probe comprises a tag or label; and wherein detecting the hybridisation complex comprises:
(a) separating un-hybridised probe from the sample; and
(b) capturing the tag or detecting the label in the sample;
wherein the presence of the tag or label is indicative of the presence of the hybridisation complex;
or wherein the probe comprises reporter and quencher fluorophores; and wherein the detection step comprises:
(a) separating un-hybridised probe from the sample;
(b) cleaving the hybridised probe to separate the reporter and quencher fluorophores; and
(c) detecting a fluorescent signal or detecting a change in a fluorescent signal;
wherein said fluorescent signal, or change in fluorescent signal, is indicative of the presence of the amplification product.

93. A method according to claim 84, further comprising performing a method for detecting S. enterica subsp. IIIa and/or IIIb on the sample.

94. A method according to claim 93, wherein said method for detecting S. enterica subsp. IIIa and/or IIIb comprises:
(a) contacting the sample with a pair of forward and reverse oligonucleotide primers, wherein said forward and reverse primers hybridise to target nucleic acid sequences located within the lacZ gene of S. enterica subsp. III, or the complement thereof;
(b) extending said forward and reverse primers to generate an amplification product, and
(c) detecting the amplification product;
wherein the nucleotide sequence of the lacZ gene of S. enterica subsp. III has at least 95% identity to a nucleotide sequence selected from SEQ ID NOs: 1, 11 or 12.

95. A method according to claim 93, wherein the method for detecting S. enterica subsp. I in the sample and the method for detecting S. enterica subsp. IIIa and/or IIIb in the sample are carried out substantially simultaneously;
wherein the method comprises the steps of:
(a) contacting the sample with:
(i) a pair of forward and reverse oligonucleotide primers, wherein said forward and reverse oligonucleotide primers hybridise to target nucleic acid sequences located within the hilA gene of S. enterica subsp. I, or the complement thereof; and
(ii) a pair of forward and reverse oligonucleotide primers, wherein said forward and reverse oligonucleotide primers hybridise to target nucleic acid sequences located within the lacZ gene of S. enterica subsp. III, or the complement thereof;
(b) extending said hilA forward and reverse oligonucleotide primers to generate a hilA amplification product, and extending said lacZ forward and reverse oligonucleotide primers to generate a lacZ amplification product; and
(c) detecting said amplification products.

96. A method according to claim 93, wherein the method for detecting S. enterica subsp. I in the sample and the method for detecting S. enterica subsp. IIIa and/or IIIb in the sample are carried out sequentially.

97. An in vitro method for quantitating S. enterica subsp. I bacterial load in a sample of interest, comprising:
(a) carrying out a detection method according to claim 84 on said sample of interest;
(b) carrying out said method on a test sample of pre-determined known S. enterica subsp. I bacterial load; and
(c) comparing the amount of amplification product detected from the sample of interest with the amount of amplification product detected from the test sample, thereby quantitating S. enterica subsp. I bacterial load in the sample of interest.

98. An in vitro method according to claim 97, further comprising quantitating S. enterica subsp. IIIa and/or IIIb bacterial load in the sample.

99. An in vitro method of determining the efficacy of an anti-S. enterica subsp. I drug over the course of a period of therapy, comprising:
(a) carrying out a detection method according to claim 84 on a first sample obtained at a first time point within or prior to the period of therapy;
(b) carrying out said method on one or more samples obtained at one or more later time points within or after the period of therapy; and
(c) comparing the amount of amplification product detected from the first sample with the amount of amplification product detected from the one or more later samples, thereby determining drug efficacy over the course of the period of drug therapy, wherein a reduction in the quantity of amplification product detected from the one or more later samples, as compared with the quantity of amplification product detected from the first sample, indicates efficacy of the drug against S. enterica subsp. I.

100. An in vitro method according to claim 99, further comprising determining the efficacy of an anti-S. enterica subsp. IIIa and/or IIIb drug over the course of said period of therapy.

101. An in vitro method of determining the efficacy of a vaccine against S. enterica subsp. I infection, comprising:
(a) carrying out a detection method according to claim 84 on a first sample obtained from a patient at a first time point prior to vaccination;
(b) carrying out said method on a sample obtained from said patient at one or more later time points following vaccination and challenge with S. enterica subsp. I bacteria; and
(c) comparing the amount of amplification product detected from the first sample with the amount of amplification product detected from the one or more later samples, thereby determining vaccine efficacy, wherein a reduction in the quantity of amplification product detected from the one or more later samples, as compared with the quantity of amplification product detected from the first sample, indicates efficacy of the vaccine against S. enterica subsp. I infection.

102. An in vitro method according to claim 101, further comprising determining the efficacy of a vaccine against S. enterica subsp. IIIa and/or IIIb infection.

103. A forward oligonucleotide primer that hybridises to a target nucleic acid sequence, which target nucleic acid sequence comprises a nucleotide sequence that is at least 75% identical to SEQ ID NO: 15 or 16, or a fragment thereof having at least 15 consecutive nucleotides thereof.

104. A reverse oligonucleotide primer that hybridises to a target nucleic acid sequence, which target nucleic acid sequence comprises a nucleotide sequence that is at least 75% identical to a nucleotide sequence of SEQ ID NO: 17 or 18, or a fragment thereof having at least 15 consecutive nucleotides thereof.

105. A set of forward and reverse oligonucleotide primers, comprising a forward primer that hybridises to a target nucleic acid sequence, which target nucleic acid sequence comprises a nucleotide sequence that is at least 75% identical to SEQ ID NO: 15 or 16, or a fragment thereof having at least 15 consecutive nucleotides thereof; and/or a reverse primer that hybridises to a target nucleic acid sequence, which target nucleic acid sequence comprises a nucleotide sequence that is at least 75% identical to a nucleotide sequence of SEQ ID NO: 17 or 18, or a fragment thereof having at least 15 consecutive nucleotides thereof.

106. A set of primers according to claim 105, further comprising one or more primers for detecting S. enterica subsp. IIIa and/or IIIb.

107. A set of primers according to claim 106, wherein said one or more primers for detecting S. enterica subsp. IIIa and/or IIIb are selected from:
(a) a forward primer comprising a nucleotide sequence having at least 75% identity to a nucleotide sequence of SEQ ID NO: 4, or a fragment thereof comprising at least 15 consecutive nucleotides thereof; and/or
(b) a reverse primer comprising a nucleotide sequence having at least 75% identity to a nucleotide sequence of SEQ ID NO: 5, or a fragment thereof comprising at least 15 consecutive nucleotides thereof.

108. An oligonucleotide probe that hybridises to a target nucleic acid sequence, which target nucleic acid sequence comprises a nucleotide sequence that is at least 75% identical to a nucleotide sequence of SEQ ID NO: 23, or a fragment thereof having at least 18 consecutive nucleotides thereof.

109. A set of oligonucleotide probes comprising a probe according to claim 108, and further comprising a probe for detecting S. enterica subsp. IIIa and/or IIIb.

110. A set of probes according to claim 109, wherein the probe for detecting S. enterica subsp. IIIa and/or IIIb comprises a nucleotide sequence having at least 75% identity to a nucleotide sequence of SEQ ID NO: 7, or a fragment thereof comprising at least 17 consecutive nucleotides thereof.

111. A kit for detecting S. enterica subsp. I in a sample, said kit comprising a set of forward and reverse oligonucleotide primers according to claim 105; and further comprising reagents for amplification of a S. enterica subsp. I-specific nucleic acid sequence; and/or reagents for detection of the amplification product.

112. A kit according to claim 111, further comprising:
(a) reagents for amplification of a S. enterica subsp. IIIa and/or IIIb-specific nucleic acid sequence; and/or
(b) reagents for detection of the amplification products.
